(19) United States
(12) Patent Application Publication
(10) Pub. No.: US 2006/0094046 A1
(43) Pub. Date: May 4, 2006

(54) Compositions and methods relating to angiogenesis and tumorigenesis

(76) Inventors: Arie Abo, Oakland, CA (US); Robert A. Stull, Alameda, CA (US); Daniel J. Chin, Foster City, CA (US); Stephen G. Osborn, Belmont, CA (US); Scot Free Kennedy, San Francisco, CA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 11/251,687

(22) Filed: Oct. 17, 2005

Related U.S. Application Data
(63) Continuation of application No. 11/056,599, filed on Feb. 11, 2005, now abandoned.
(60) Provisional application No. 60/543,793, filed on Feb. 11, 2004.

(51) Int. Cl. C12Q 1/68; C12M 1/34

(52) U.S. Cl. 435/6; 435/287.2

(57) ABSTRACT

Methods for identifying nucleic acid molecules and polypeptides that participate in angiogenesis and tumorigenesis, and associated methods and products are provided.

### CROSS REFERENCE TO RELATED APPLICATIONS

This application is a continuation of U.S. patent application Ser. No. 11/056,599, entitled “Compositions and Methods Relating to Angiogenesis and Tumorgenesis,” filed Feb. 11, 2005, which claims the benefit under 35 U.S.C. § 119, of U.S. Provisional Patent Application Ser. No. 60/543,793, entitled “Compositions and Methods Relating to Angiogenesis and Tumorigenesis,” filed Feb. 11, 2004. The above referenced are incorporated by reference herein in their entirety.

### FIELD OF THE INVENTION

The invention relates to methods for identifying nucleic acid molecules and polypeptides that participate in angiogenesis and tumorigenesis. The invention also relates to nucleic acid molecules and polypeptides identified according to the teachings of the invention. The invention also relates to methods for using the nucleic acid molecules and polypeptides of the invention, e.g., as biomarkers, therapeutics and targets for therapeutics.

### BACKGROUND OF THE INVENTION

The process of tumorigenesis has long been recognized to depend upon complex interactions of a tumor with its non-transformed tissue environment (Paget 1889). Beyond transformation and increased proliferation, many pathways are activated both in the growing tumor and its environment to culminate in an established solid tumor. For example, adhesive pathways are activated to enable transformed cells to aggregate and form a microtumor. Subsequently, microtumors must avoid destruction by the immune system and elicit vasculature formation for continued growth (Hong et al 2003, Bergers et al 2003). It is thought that primary or metastatic microtumors about 1 mm³ in size are metastable; they are either (i) resolved by the immune system, (ii) remain in a steady-state with balanced proliferation and apoptosis or (iii) undergo aggressive growth as long as a vasculature is developed to provide nutrients to the growing mass (Fidler 2003).

In support of these events, cell-matrix adhesion proteins, cell surface antigens, angiogenic factors and modulatory agents have been found to be differentially expressed in several experimental models of tumorigenesis (Glinsky et al 2003, Pedersen et al 2003, Creighton et al 2003) and in tumor biopsy samples relative to control tissues (Perou et al 2000, Dhanasekaran et al 2001). Experimental models with established tumorigenic human cell lines have compared the gene expression profiles between the cultured parental cells and after implantation into immune-deficient murine hosts (Creighton et al 2003).

The extent of vascularization to support an established tumor will vary according to the tumor type and tissue environment as a result of variable levels of proteases, receptors or regulators of pericyte and/or endothelial migration, proliferation, and differentiation (Holash et al 1999, Bergers et al 2003). Additionally, some tumors such as early grade astrocytomas can leverage existing normal brain blood vessels without substantial vasculogenesis for subsequent angiogenic sprouting of new vessels from preexisting vessels (Vajkoczy et al 2002). Further, vascularization depends upon a tuned interaction in the tissue microenvironment between endothelial cells and pericytes (Benjamin et al 1998, Gerhardt et al 2003). Vascularization of solid tumors may also be heterogeneous with a rapidly growing margin surrounding a hypoxic core following regression of co-opted vessels that supported early tumor growth (Holash et al 1999). Complicating this picture is the potential for ‘vascular mimicry’ where breast tumor derived cells express many endothelial markers and may serve as rudimentary channels (Shirakawa et al 2002).

Many angiogenesis studies have used cultured primary vascular endothelial cells and shown the significant roles of VEGF, FGF, PDGF, chemokines and cell-matrix adhesion proteins (Aonuma et al 1998, Hattori et al 2001, Bergers et al 2003). These assays for endothelial cell migration include the chorioallantoic membrane (Ekstrand et al 2003), matrigel migration assays (Maeshima et al 2000) or 3D-collagen assays (Mallett et al 2003). However, the limits of studying the angiogenic process with established endothelial cells in vitro have been recognized. Tumorigenesis involves both heterophilic and homophilic cellular communication and adhesion between not only endothelial cells but also pericytes and smooth muscle cells; hence other cell surface proteins and secreted factors are absent from such assays (Bergers et al 2003).

A search for tumorigenic genes common to tumors of diverse origin should be as broad as possible and hence should not be limited to a single tumor type or tissue source. In the present invention, the search for tumorigenic genes was examined with a more focused approach with respect to the transcripts as well as a broader survey by examining multiple tumor sources in order to identify differential genes common to multiple solid tumors.

### BRIEF DESCRIPTION OF THE FIGURES

FIG. 1. Gene ontology of custom chip probes. The ontological classification of 3531 cell surface or secreted genes was extracted from the Gene Ontology at the third level. Genes lacking GO annotations at this level were derived from the second level.

FIG. 2. Principal components analysis of array data. Pooled tumor data were compared to pooled parental cell line data. The first 3 principal components of the analysis are shown from the best vantage point to show separation of the three classes. Open circles re the parental cell lines, “X” denotes the various xenograft tumors, and the small solid dots are the reference cDNA sample (derived from the Universal RNA) co-hybridized with all experimental samples. The cell lines corresponding to the various tissue sources of the parental cell lines were: Ovary, SKOV3; Prostate, PC3; Breast, MDA MB-231; Colon, HCT116; and Lung, A549.

FIG. 3. Plot of linear discriminant profile of 70 probes that distinguish xenograft tumors from parental cell lines. The top 70 PCA coefficients along the third component were selected; these 70 probes correspond to 54 genes. Positive values indicate “Xenograft tumor” while negative values indicate “Parental Cell line”. FIG. 3A: The x-axis shows either numbered tumor (top) or parental cell (bottom) samples and the y-axis is an arbitrarily scaled output reflecting the accuracy in assigning a sample as a xenograft tumor or parental cell line. FIG. 3B: A graphical reation of the LD-54 genes expression profiles. For genes with multiple probes, the highest value is shown. Classified by a non-redundant filtering of the Gene Ontology biological process terms, the genes are shown with a color scale reing relative fold induction to pooled parental cell line data. The left-most color column designated by ‘X’ is the average ratio, while the remaining five columns correspond to Colon (HCT116), Breast (MDA MB-231), Lung (A549), Prostate (PC3) and Ovarian (SKOV-3) carcinoma xenografts respectively.

FIG. 4. Ontological classification of 175 genes derived from three analyses. The 149 genes derived from the ANOVA analysis of pooled xenograft versus pooled parental cell line data, the 54 genes identified by the linear discriminant analysis and the 12 genes derived from the intersect of ANOVA of individual tumors are shown. Gene Ontology terms were extracted at level 3 for the Unigene gene names. Twenty genes lacking level 3 GO terms were annotated with level 2 terms, and sixteen genes were manually curated from the literature or annotated as ‘not known’. FIG. 4A: Molecular function classification, FIG. 4B: Biological process classification.

FIG. 5. Comparison of differential expression of genes in parental cells versus reference cDNA synthesized from universal RNA (left) and pooled tumors versus parental cell lines (right). Genes differentially expressed in the parental cells relative to the reference cDNA were analyzed by a 2-way ANOVA (P_{corr}<0.001). A subset of the genes are shown. The corresponding cognate tumors with differential expression at a 99.9% confidence are shown. The heat maps indicate relative fold-induction or suppression in a linear color-encoded scale shown at the bottom. Mean ratios are indicated by X, C=colon, B=breast, L=lung, P=prostate, O=ovary.

FIG. 6. Quantitative PCR analysis of selected genes. Two tumors of each tumor type were analyzed by quantitative PCR. The measured fold change relative to cell line was determined. RNA amounts per well being normalized by beta-actin signal. In general <2-fold changes are not significant. Hence a call of 1.5 fold down may not actually differ from 1.5 up. Specific tumor types are indicated by the first initial followed by the tumor number: i.e., C1=colon tumor #1, O1=ovary tumor #1, L1=lung tumor #1, B1=breast tumor #1, P1=prostate tumor #1.

FIG. 7. Overlap of differentially expressed genes identified by three analyses: ANOVA-p149=149 genes derived from the ANOVA analysis of pooled data, LD-p54=linear discriminant list of 54 genes from pooled data, and ANOVA-il2=twelve genes resulting from a comparison of differentially expressed genes from the ANOVA analysis of individual tumors compared to parental cell lines. The heat maps indicate relative fold-induction or suppression in a linear color-encoded scale shown at the bottom. Mean ratios are indicated by X, C=colon, B=breast, L=lung, P=prostate, O=ovary.

### DESCRIPTION OF THE INVENTION

The invention provides methods for identifying nucleic acid molecules and polypeptides that participate in angiogenesis and tumorigenesis. The invention also provides nucleic acid molecules and polypeptides identified according to the teachings of the invention. The invention also provides methods for using the nucleic acid molecules and polypeptides of the invention, e.g., as biomarkers, therapeutics and targets for therapeutics.

A custom oligonucleotide microarray was designed containing probes for all publicly known and putative secreted and cell surface genes. The custom oligonucleotide microarray was used to analyze five diverse human transformed cell lines and their derivative xenograft tumors. The origins of these human cell lines were lung (A549), breast (MDA MB-231), colon (HCT-116), ovarian (SK-OV-3) and prostate (PC3) carcinomas. Three different analyses were performed: (1) A PCA-based linear discriminant analysis identified a 54 gene profile characteristic of all tumors when pooled tumor data were analyzed, (2) application of MANOVA (P_{corr}<0.05) to pooled tumor data revealed a larger set of 149 differentially expressed genes, and (3) after MANOVA was performed on data from individual tumors, a final comparison of differential genes among all tumor types, revealed 12 common differential genes. Seven of the 12 genes were identified by all three analytical methods. These included late angiogenic, morphogenic and extracellular matrix genes such as ANGPTL4, COL1A1, GP2, GPR57, LAMB3, PCDHB9 and PTGER3. The differential expression of ANGPTL4 and COL1A1 and other genes was confirmed by quantitative PCR. Overall, a comparison of the three analyses revealed an expression pattern indicative of late angiogenic processes.

In one aspect, the invention relates to a method for designing a custom microarray to study the expression profiles of a specific set of genes; e.g., the method for designing a custom microarray to study the expression profiles of all publicly known and putative secreted and cell surface genes. In another embodiment, the invention relates to the resulting custom microarray, e.g., the custom microarray comprising probes for over 3000 genes encoding secreted and cell surface polypeptides.

Another aspect of the invention relates to an experimental model of tumorigenesis and angiogenesis. In the experimental model of the invention, a xenograft tumor is prepared from a cancer cell line, as described below. The expression profiles are obtained using a microarray comprising probes for certain nucleic acid molecules. A variety of statistical methods are used to identify polynucleic acid molecules that are differentially expressed in the xenograft tumors relative to the parental cell lines. In a further embodiment, differential expression of certain nucleic acid molecules in parental cells versus reference cDNA synthesized from universal RNA is also analyzed.

Another aspect of the invention relates to nucleic acid molecules identified as differentially expressed using the experimental model of the invention. Such nucleic acid molecules may be deoxyribonucleic acid molecules or ribonucleic acid molecules. Such nucleic acid molecules may be single stranded or double stranded. In one embodiment, the nucleic acid molecules are those included in the 54-gene set derived from the linear discriminant analysis (LD-p54), described below and set forth in Table 1 and Table 2. In another embodiment, the nucleic acid molecules are those included in the 149-gene set derived from ANOVA analysis (ANOVA-p149), described below and set forth in Table 2. In another embodiment, the nucleic acid molecules are those included in the 12-gene set resulting from the comparison of differentially expressed genes from the ANOVA analysis of individual tumors compared to parental cell lines (ANOVA-i12), described below and set forth in Table 2. Another aspect of the invention relates to fragments of the nucleic acid molecules of the invention, modified nucleic acids molecules of the invention, molecules that hybridize to nucleic acid molecules of the invention and molecules that comprise the nucleic acid molecules of the invention.

Another aspect of the invention relates to the polypeptides that are encoded by the nucleic acid molecules of the invention. Included within this aspect of the invention are fragments of the polypeptides of the invention, modified polypeptides of the invention, and molecules that comprise the polypeptides of the invention such as fusion proteins. Precursors of a polypeptide of the invention, metabolites of a polypeptide of the invention, a modified polypeptide of the invention and a fusion protein comprising all or a portion of a polypeptide of the invention are included in this aspect of the invention.

Another aspect of the invention relates to antibodies, antibody fragments, or other molecules that specifically recognize and bind to a polypeptide of the invention. Such molecules can be used, for example, in methods for detecting polypeptides of the invention, or in methods for treatment of cancer or other disease.

Another aspect of the invention relates to methods for determining the concentration of a polypeptide of the invention, detecting the presence of a polypeptide of the invention, or determining the activity of a polypeptide of the invention. For example, the presence of a polypeptide of the invention can be determined using an enzyme-linked immunosorbent assay (ELISA) comprising an antibody that specifically recognizes a polypeptide of the invention. Methods for detecting the concentration, presence or activity of a polypeptide of the invention could be used in the diagnosis, staging, imaging or other characterization of a cancer or other disease.

Another aspect of the invention relates to methods for treatment of a cancer or other disease. The basis for such methods for treatment are known in the art and typically comprise inhibition or inactivation of a polypeptide of the invention, inhibition of translation or transcription of a nucleic acid molecule of the invention. Some methods are based on inactivation of the proteins by antibodies inhibitors. Other methods involve using the nucleic acids of the invention to compensate for defective genes (gene therapy).

Another aspect of the invention relates to compositions comprising a polypeptide or nucleic acid molecule of the invention, or an inhibitor of, an antibody to or a modulator of a polypeptide or nucleic acid of the invention. Such compositions may be pharmaceutical compositions in which the polypeptide or nucleic acid molecule, or the inhibitor, antibody or modulator, is formulated for introduction into the body as a therapeutic.

The scientific basis for the compositions and methods described above as aspects of the invention are well-known in the art and such compositions and methods are enabled by differential gene expression data, as disclosed herein (Salceda et al 2003).

In the experimental tumorigenesis model of the invention, the attachment and growth of a micro- or metastatic tumor was examined using human xenograft tumors in nude mice. The end-point of a xenograft assay is the formation of a solid tumor, and thus genes supporting vasculogenesis and angiogenesis are likely differentially expressed in a xenograft tumor relative to the parental cell lines that were adapted to culture in vitro.

In order to find common tumorigenic genes regardless of tissue origin, a panel of 5 adenocarcinoma cell lines was used from breast, colon, and lung, ovarian and prostate tumors was used. These cell lines reproducibly yield solid tumors in a standard xenograft assay in immuno-compromised mice (Giard et al 1973, Cailleau et al 1974, Kaighn et al 1979). While there may be individual differences in capillary branching or density between tumor types, the xenograft assay requires vascular development to support solid tumor formation in a relatively avascular subcutaneous site.

According to a strategy of the invention, the expression profiles of secreted and cell surface genes from five different tissue sources were compared. Multiple tumors were derived from each parental cell line to examine the potential for tumor heterogeneity arising from the primary isolate, but relatively consistent behavior was found within any tumor group. However, tumor-specific genes for each tumor type were found while a profile of genes shared amongst all tumor types by multiple analytical approaches was identified. Overall, the results comprise a foundation of commonly regulated tumorigenic genes across tissues such as fundamental angiogenic inducers and regulators.

Because the early tumorigenic events largely rely upon secreted factors, cell surface receptors or integral membrane proteins, a strategy of the invention was to employ a custom microarray to focus on the expression of genes chosen on the basis of their cellular localization. Hence, an experimental microarray strategy was implemented with high replication and coverage of all possible secreted and cell surface proteins. Also, focusing on all known and predicted cell surface and secreted genes allowed the design of more intra-chip replicates for improved data reliability. While prioritizing on the ‘Function’ category of the Gene Ontology (see the Gene Ontology web site), the range of ‘Biological Processes’ covered by the gene selection remained broad. In contrast to early concerns that a sub-selection of genes might result in a systemic bias, relatively small numbers of genes were found to be common to all xenograft tumors due to the robust experimental design and statistical analysis.

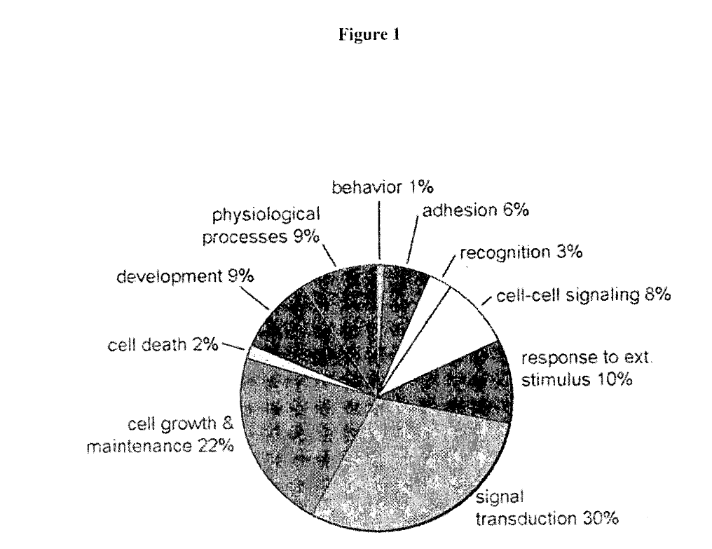

A custom oligonucleotide microarray was developed to focus on an ontologically restricted set of secreted and cell surface genes for higher data reliability using a matrix design with intra-chip replicates in addition to replicate chips. Due to the limits of the Gene Ontology classification, multiple strategies had to be used to derive a relatively complete collection of secreted and cell surface genes. For example, some proteins have multiple localization sites on the basis of newer experimental evidence absent from curated databases; e.g., SORCS3, HDGF. For such genes with multiple cellular localizations, the literature (PubMed, NCBI) was the annotation source for finding other secreted and cell surface proteins. Finally, other putative secreted and transmembrane-encoding genes and exons were analyzed from hypothetical predictions from the UCSC Human Genome. Redundant genes were removed by a combination of blastn/blastp comparisons and manual curation, but many putative membrane-encoding exons of potential proteins were included. A final tally of 3531 genes was composed of 1057 secreted genes, 1338 G-protein coupled receptor (GPCR) genes with the remainder classified as various integral membrane proteins and cell surface proteins. An ontological view of the custom chip's content is shown in FIG. 1.

In consideration of potential global changes of a selected set of genes, numerous positive and negative controls were included in the array design; including genes characteristic of some tumors (e.g. the estrogen receptor for a subset of breast tumors) and many ‘housekeeping’ transcripts (e.g. b-actin) commonly used to normalize quantitative PCR-studies. However, co-hybridizing all samples with a reference cDNA derived from a mixture of up to 10 human cell lines enabled ‘normalization’ with respect to feature, chip, and dye for the MANOVA analysis. This strategy minimizes the potential concern for a skewed normalization by a sub-selected gene population or possible differential behavior of the included ‘housekeeping’ genes in the xenograft tumors.

Several multivariate analyses of the microarray data were performed to find characteristic tumorigenic genes. The microarray analysis of variance (MA-ANOVA) tools (Kerr et al 2001) were chosen for their sensitivity and robustness in measuring differential expression versus previous T-test and log-ratio methods using thresholds for induction or suppression. This was particularly important in these studies that used a relatively complex design with on-chip and inter-chip probe replication, multiple tumor samples and tumor types, dye-swap and a common reference RNA sample for all hybridizations. Thus, this strategy helps avoid any systematic bias from using a chip containing probes for only secreted and cell surface genes.

A custom database was developed (Osborne et al 2003) to allow dynamic re-grouping of data to facilitate multiple analytical models such as pooled tumor data or individual tumor types and their parental cell lines.

Initially, the differentially expressed genes were identified in all tumors relative to all parental cells regardless of tissue origin. Hence, all the xenograft data were pooled into a single dataset and compared to the pooled parental cell line data. Similarly, both the pooled tumor and pooled parental cell line data were compared to the pooled reference cDNA hybridization data. These data were analyzed by both principal components analysis (PCA) and multivariate analysis of variance (MANOVA).

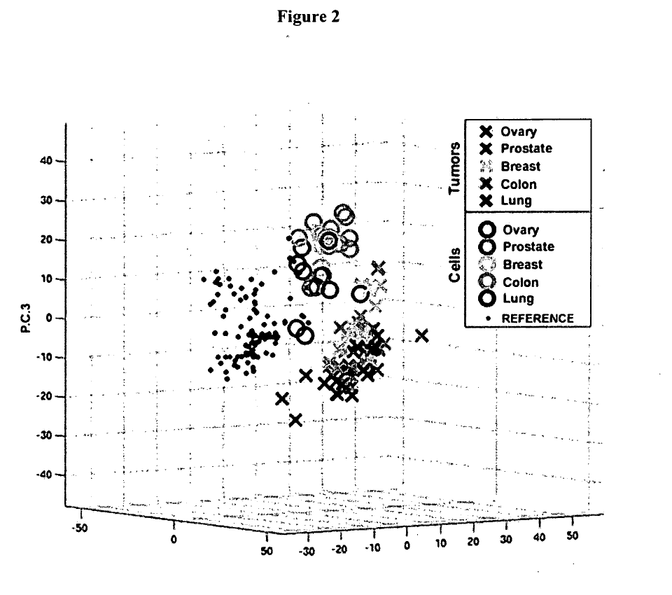

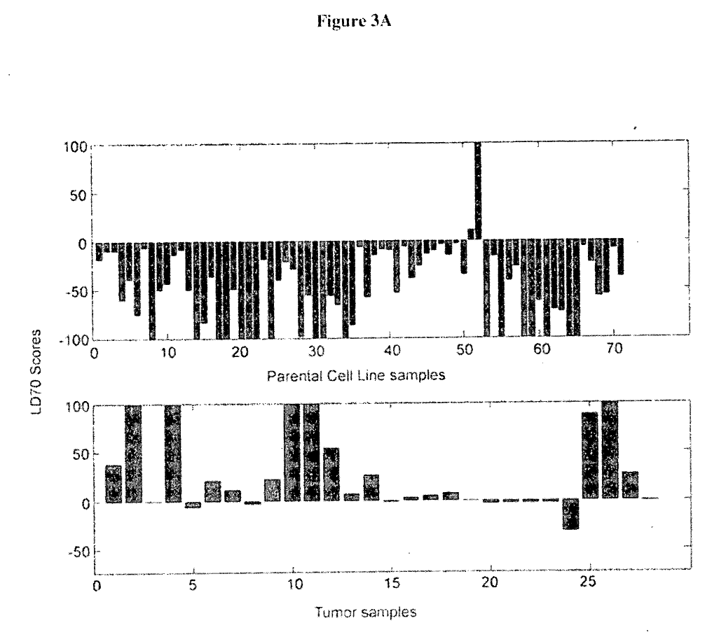

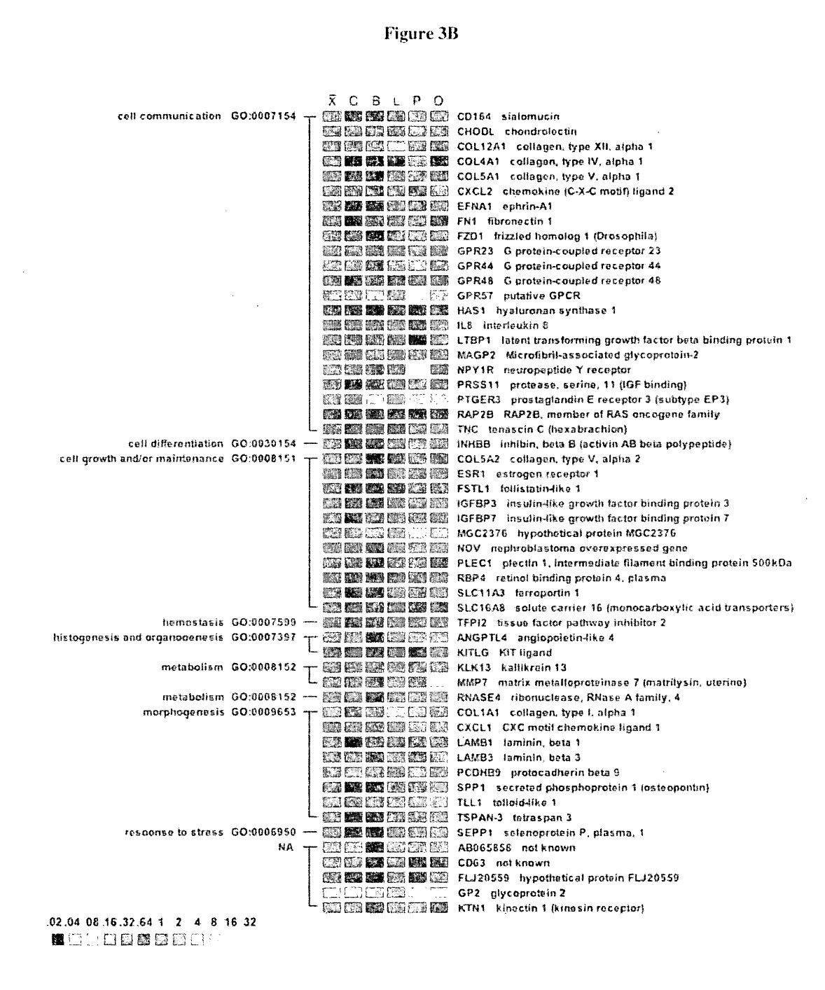

PCA was used both as a general overview and quality control for the pooled data. Even with unprocessed data not normalized by the universal RNA reference sample, a clear separation between pooled parental cell data and the pooled tumor data was seen. FIG. 2. To further analyze the data, versions of the principal component with the highest correlation to sample type were iteratively ‘trimmed’ and tested to determine their accuracy in assigning samples to either the tumor or cell line categories. This analysis retained 70 of the largest coefficients and res a simple linear discriminant (LD) of 70 probes that corresponds to 54 genes that fairly accurately distinguishes between the two sample types of parental cell lines and xenografted tumors, FIG. 3A. ‘Leave-one-out’ testing, where each of the 99 samples was removed in separate analyses, generated a profile that was 79-80% accurate in predicting a tumor. The same method applied to 1000 label-permuted datasets never exceeded 65% accuracy with a median and minimum accuracy of 49% and 39.3% respectively. This suggests that the gene profile generated by the analysis of the invention can distinguish between the pooled xenograft data and the pooled cell line data in a verifiable manner.

The 54-gene profile derived from the linear discriminant (LD-p54) was distributed amongst numerous biological processes using the Gene Ontology classification terms. Table 1 lists the Gene Ontology classification of 54 genes identified by a linear discriminant. A ‘level 3’ annotation of the biological process Gene Ontology terms was applied to the list. Many genes were classified in multiple biological process categories as a result of their biological complexity; e.g., fibronectin (FN1) is classified into 8 biological processes including cell motility, response to stress, cell communication, response to external stimuli, extracellular matrix structural constituent, protein binding and glycosaminoglycan binding. Other genes are involved with cell adhesion or extracellular matrix, cellular growth or the regulation of cellular proliferation, various membrane proteins with known or inferred functions, transporters or channels, and proteases or protease inhibitors. A non-redundant ontological classification of the genes identified by the linear discriminant is shown with a graphical reation of their behavior across all tumor types in FIG. 3B.

While most genes are upregulated in xenograft tumors, other genes are uniformly suppressed; e.g., hyaluronan synthase 1 (HAS1), RAP2B, a member of the RAS oncogene family and solute carrier 16 (SLC16A8), an organic ion transporter. Because the linear discriminant analysis uses a weighted sum, not all of the identified genes behave consistently across all xenograft tumors; e.g., CD164 or COL4A1. CD164 is a sialomucin and has been found modestly elevated in many colon and prostate carcinomas (Su et al 2001). Consistent with the results using the xenograft model of the invention, collagen IV alpha 1 was suppressed in 7 of 7 established colon cell lines, suppressed in 5 of 9 lung cell lines (Ross et al 2000).

Cell adhesion and extracellular matrix genes were also in the LD-54 gene profile. The cell adhesion genes could be involved with heterophilic or homophilic adhesion such as chondrolectin (CHODL) and protocadherin beta 9 (PCDHB9). The extracellular matrix genes were comprised of five collagen genes (COL1A1, COL4A1, COL5A1, COL5A2 and COL12A1), microfibrillar glycoprotein 2 (MAGP2), cartilage matrix protein (MATN1) and tissue factor pathway inhibitor 2 (TFP12). Also in the profile was osteopontin (SPP1), normally a secreted extracellular matrix protein, which is soluble when derived from tumors (Rittling et al 2003) and acts as a cytokine to induce both neovascularization and angiogenesis (Hirama et al 2003, Leali et al 2003). Consistent with previous reports that found COL1A1 to be induced in most breast carcinomas (Perou et al 1999, Su et al 2001) and a subset of ovarian and colon carcinomas (Su et al 2001), COL1A1 expression was found to be elevated in each of the tumors examined using the xenograft model of the invention. In contrast to the modest induction or reductions in SPP1 found herein, SPP1 was found strongly induced in kidney cancer cell lines (Ross et al 2000), kidney carcinomas (Su et al 2001), and ovarian and lung carcinomas (Su et al 2001).

The pooled data was also subjected to ANOVA using the two broad classifications of parental cells and xenograft tumors. This analysis identified 156 probes reing 149 differentially regulated genes at the 99.9% confidence level. See Table 2.

Table 2 is the merged list, of genes identified by three analyses: (a) ANOVA of pooled xenograft data versus pooled parental cell lines yielded 149 differential genes (Ap), (b) Linear discriminant analysis of the pooled data identified 54 genes (LD) and (c) ANOVA of individual xenograft tumors compared to their individual parental lines were compared to yield a consensus of 12 genes, (Ai). For each gene identified by the analyses, its presence is denoted by ‘1’ and its absence noted by ‘0’. The pooled maximum MANOVA p-value is reported along with the aggregate ratio. For genes with multiple independent probes, the probe reporting the maximum p-value is shown. Seven genes common to all three lists are highlighted in yellow. Twenty-nine genes identified by both the ANOVA-p149 and are highlighted in green. Three genes found in only the ANOVA-p149 and ANOVA-i12 lists are shown in blue.

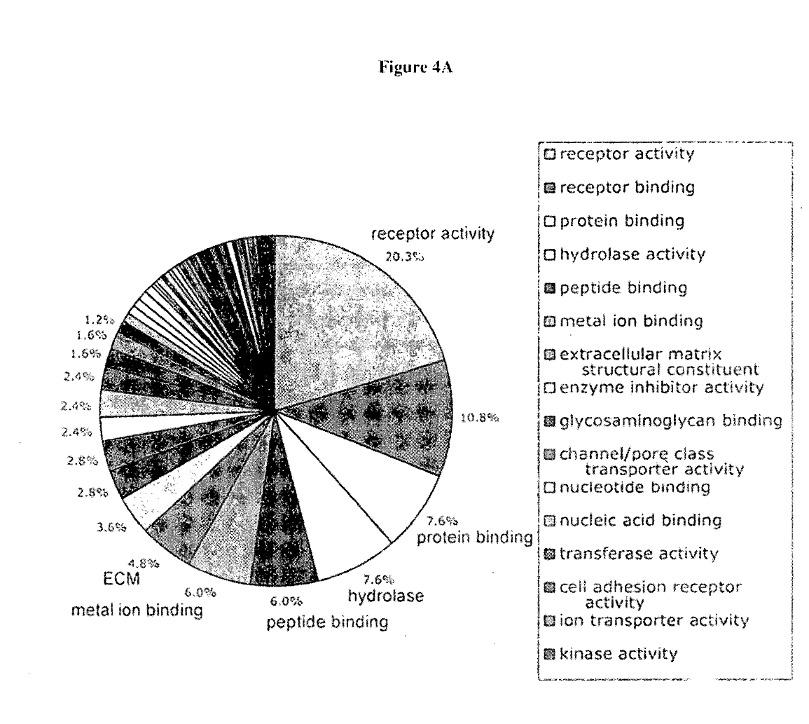

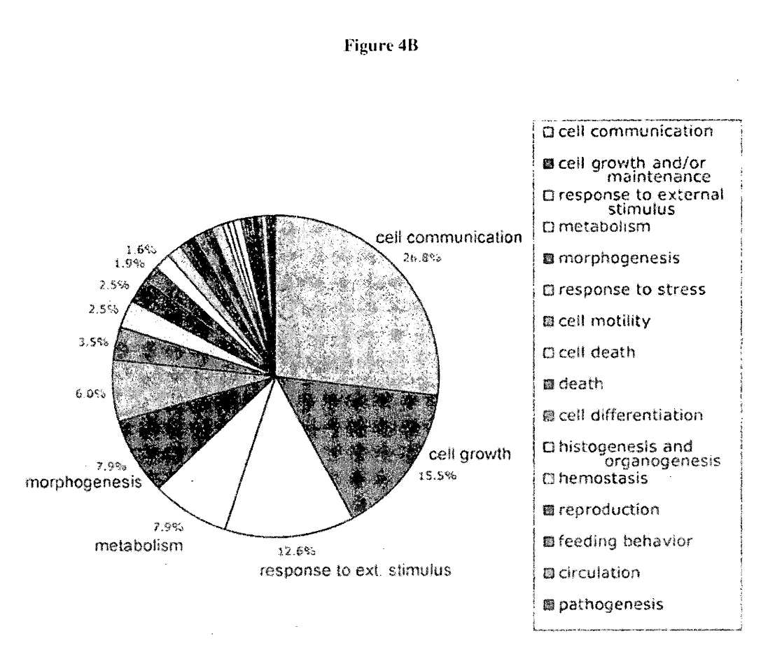

The range of induction or suppression of this set of genes (ANOVA-p149) was 6-fold induction and 5-fold suppression. Twenty-nine of the 54 genes found by the above linear discriminant analysis were found in the list of 149 ANOVA-qualified probes. An ontological clustering of the ANOVA-p149 genes revealed patterns of proteases and protease inhibitors, cell-matrix adhesion genes, receptors, ion channels, various ligands including chemokines and interleukins, additional angiogenic genes and several genes of unknown function; the major ontological groups are shown in FIG. 4. Of the angiogenic genes found in the ANOVA analysis of pooled data, angiopoietin2 (ANGPT2-2.2-fold elevated, P_{corr}<0.003) and the prostaglandin E receptor 3 (PTGER3-6.4-fold, P_{corr}<0.001) are of note since ANGPT2 and VEGFA play critical roles in early angiogenesis (Zagzag et al 1999, Holash et al 1999). Furthermore, prostaglandins can induce VEGFA production (Harada et al 1994, Gallo et al 2001) via a hypoxia-induced pathway (Fukuda et al 2003). Coincident with these observations, IGFBP7, in both the ANOVA-p149 and LD-54 lists, modulates IGF mitogenic activity (Oh et al 1996) and stimulates prostacyclin synthesis (Yamauchi et al 1994) perhaps to take advantage of the 6-fold increased PTGER3 expression. Finally, induction of TEM5, a marker of tumor endothelial angiogenesis (Carson-Walter et al 2001), was also found significant by the ANOVA analysis of pooled data (1.37-fold, P_{corr}<0.001).

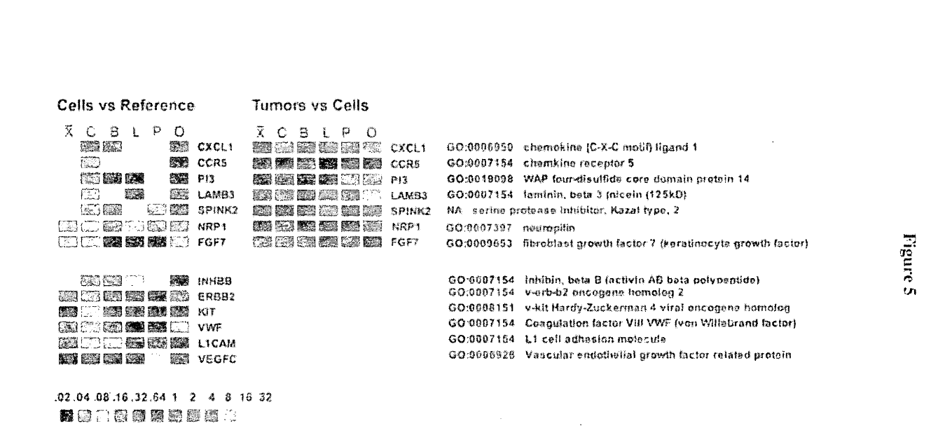

Many of the genes induced in the parental cell lines relative to the reference cDNA were still capable of further induction or they were suppressed in the xenograft tumors. Of the 861 genes that were found to be differentially expressed in the parental cell lines relative to the reference cDNA by a 2-way ANOVA (P_{corr}<0.001), several of the induced genes shown in FIG. 5 are known to be over-expressed in some carcinomas such as inhibin beta 3 (INHBB), laminin beta 3 (LAMB3), v-erb-b2 oncogene 2 (ERB-B2), and coagulation factor VIII (VWF) (Su et al 2001). While LAMB3 was induced 12.5-fold in the parental cells relative to the reference cDNA, LAMB3 was further induced 1.85-fold (P_{corr}<2e-12) in the pooled tumor data. Reciprocal behavior was also found; e.g., the alternate VEGF receptor NRP1 was induced 3.6-fold in the parental cell line relative to the reference cDNA, but NRP1 was modestly suppressed 1.3-fold (P_{corr}<0.006) in the pooled tumor data. Similarly, the serine protease inhibitor SPNK2 was induced 12-fold in the parental cell line relative to the reference cDNA but SPINK2 was suppressed 2.56-fold in the pooled tumor data (P_{corr}<0.001), FIG. 5. These results suggest a wide dynamic range of gene expression from the reference cDNA, parental cell lines and xenograft tumors.

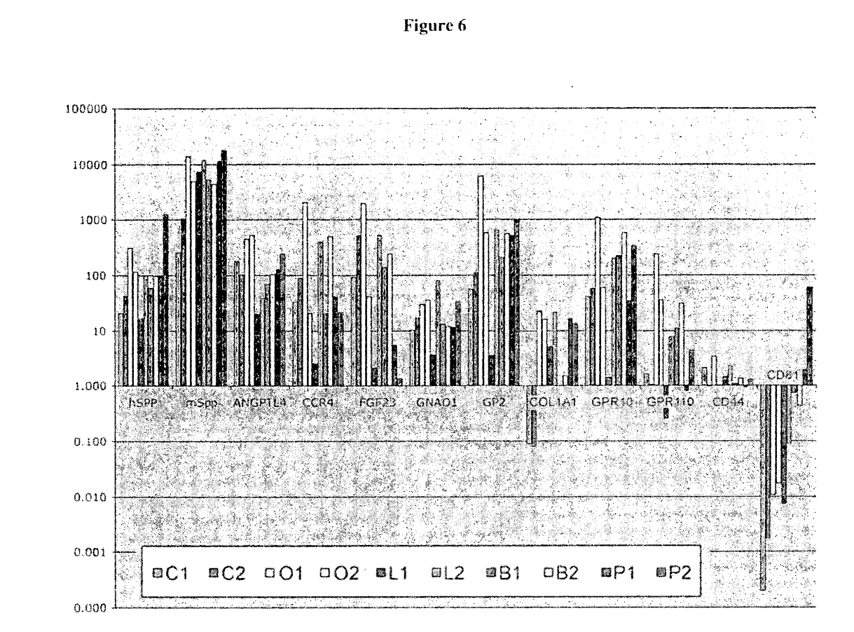

The differential expression of selected genes was confirmed by quantitative real-time PCR using the same RNA samples. The vast majority of the genes tested by RT-PCR validated the array analysis, FIG. 6. In some instances, discrepancies in fold-induction can be explained by methodological differences since the array data were all normalized to the co-hybridized universal-RNA sample, while the PCR data were normalized to a b-actin probe. Differential expression of ANGPTL4, GP2, GNAO1, CCR4, FGF23, SPP1 and COL1A1 were qualitatively consistent in both the PCR and array analyses. However, two of the down-regulated genes identified by the array analysis, both G-protein coupled receptors, were found by PCR to be elevated, albeit with large variability; GPR10 was induced 281-fold SD=469 and GPR110 induced 50-fold SD=105. Of the two down-regulated genes examined by quantitative PCR, CD81 was consistent in both assays, while CD44 was measured by PCR as unchanged or minimally induced yet array analysis indicated CD44 was suppressed. However, the aggregate 2-fold CD44 induction as measured by quantitative PCR is the threshold of what is considered significantly distinguishable from unchanged.

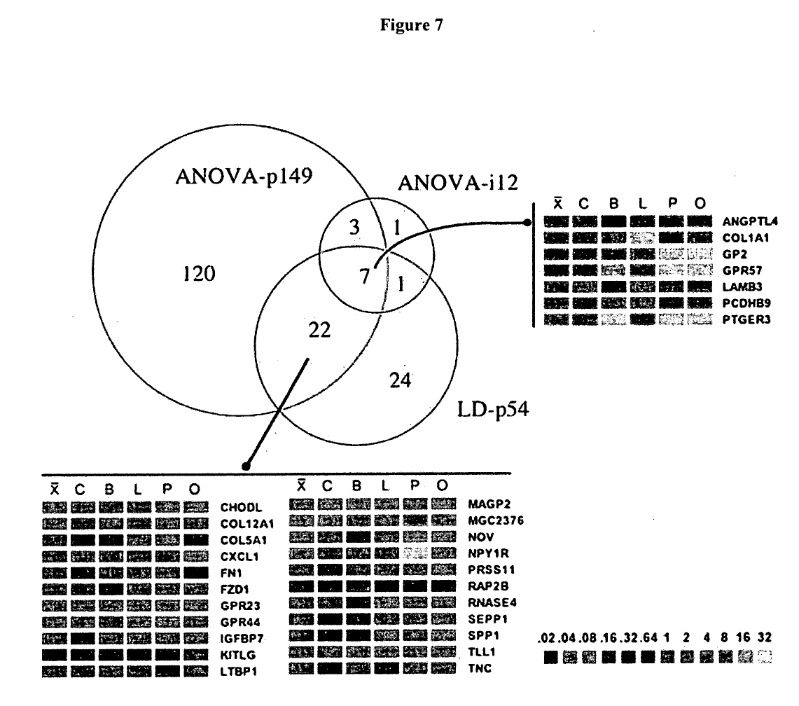

To accommodate the possibility that tumor type was an important contributor to differential gene behavior, a third analysis was performed by examining the intersection between the differential genes of each individual tumor type. For this restrictive analysis, each tumor type was simply examined relative to its parental cell line by ANOVA. Approximately 91-312 genes were differentially expressed at 99.9% confidence for each cell line: SKOV-3, 125 genes; MDA, 312 genes; HCT116, 124 genes; A549, 159 genes; and PC3, 91 genes. Twelve genes were found in common amongst these separately analyzed tumor types, ANGPLT4, COL1A1, epithelial membrane protein 3 (EMP3), GNAO1, glycoprotein 2 (GP2), GPR57, HAS1, HLA-A, laminin beta 3 (LAMB3), PCDHB9, protease inhibitor 3 (PI3), and PTGER3, Table 2 and FIG. 7. After comparing all the individual tumor ANOVA analyses, 7 of these 12 genes were identified were common to the LD-54 gene profile: ANGPLT4, COL1A1, GP2, GPR57, LAMB3, PCDHB9, and PTGER3. Eight of the 12 genes were differentially induced between 1.9 and 6.4 fold while 2 genes (PI3 and HAS1) were suppressed 1.7 and 3.6 fold. Real-time PCR analysis generally confirmed these observations in multiple tumor samples but with higher induction ratios; e.g., the level of ANGPTL4 was measured by PCR as induced 19 to 453 fold with a average fold induction of 185 SD=170 for 10 tumors (2 of each type). The aggregate induction of ANGPTL4 in the array analysis was 2.09 fold (P_{corr}<2e-9). Similarly, COL1A1 was measured by PCR as induced in most tumors with an average 9.8-fold (SD=9.1) versus a 3.64-fold induction found by microarray analysis. Finally, in ovarian and prostate tumors, angiopoietin 2 (ANGPT2) measured by PCR was elevated 6-fold versus the 2.2-fold induction found by microarray analysis.

Two of the 12 genes shared amongst the individually analyzed tumors have unknown functions or roles; GPR57 was isolated from a genomic screen and is believed to be a pseudogene (Lee et al 2001) while GP2 is a GPI-linked membrane protein secreted with zymogen granules (Fukuoka et al 1991). The remaining genes have either well-characterized functions or biological roles, particularly angiogenesis (ANGPTL4), morphogenesis (LAMB3, COL1A1, PCDHB9, or cellular mobility or communication (HAS1, PTGER3, PCDHB9, LAMB3). ANGPTL4 originally was described as an induced target of peroxisome proliferator-proliferatoractivated receptor gamma that is involved in glucose homeostasis and differentiation of adipose activated tissue (Yoon et al 200 2001). Subsequently ANGPTL4 was shown to possess angiogenic activity in the chick allochorionic migration assay (Le Jan et al 2003). More recently, ANGPTL4 was shown to bind and inhibit lipoprotein lipase (Yoshida et al 2002), a function consistent with the cachexia induced by tumors, where a reduction of fatty acid incorporation into fat cells serves the energy needs of the tumor rather than the host. ANGPTL4's angiogenic action has been reported to be independent of VEGF in a renal carcinoma model (Le Jan et al 2003) whereas endothelial ANGPT2 expression acts in concert with VEGF expression in vascular tumors to facilitate vascular remodeling ( Vajkoczy et al 2002). Further, differential tumor expression of angiopoietin 2 (ANGPT2 with 2.23-fold P_{corr}<0.005) was found by the ANOVA of pooled data. As noted above, ANGPTL4 was similarly induced (2.09 fold, P_{corr}<2e-9).

Other induced angiogenesis-related genes included a variety of cell-matrix adhesion genes or immune recognition genes. Examples of the former include COL1A1, LAMB3, and PCDHB9. Interestingly, in both the ANOVA of pooled data and the ANOVA of individual tumors, HLA-A a gene involved in antigen ation (Lopez et al 1989) was consistently suppressed in all tumors, 1.7-fold (P_{corr}<6e-7). This suggests that the survival of the original human tumors, from which the cell lines were initially isolated, resulted partly by mitigating antigen ation that would promote evasion of immune recognition.

Due to the avascular site of injection and the collection of xenografts after 28-29 days, it is not surprising to find patterns of differential gene expression that reflect a portion of the tumorigenic process rather than a preponderance of early transforming events. This conclusion is largely supported by the genes common to the three analyses, two of which are based on the analysis of pooled data. In contrast, genes known to act relatively early in vasculogenesis, such as VEGF or FGF (Aonuma et al 1998, Hattori et al 2001), were generally not significantly altered. Consistent with the lack of strong, differential VEGF expression, TIMP-3 was found to be induced, 1.4-fold (P_{corr}<0.001). TIMP-3 can block the function of VEGF2R/KDR independently of its protease inhibition site (Qi et al 2003). The strong 5-fold induction of NPY1 also supports angiogenic events downstream of VEGF since NPY1 participates in vasoconstriction (Zukowska-Grojec et al 1996) and capillary sprouting and differentiation (Lee et al 2003). Recently, the potent effect of ligand neuropeptide (NPY) upon angiogenesis was shown to yield branching vasodilated structures distinct from those generated by VEGF (Ekstrand et al 2003).

Interestingly, neuropilin 1 (NRP1) was differentially expressed (1.31 fold suppressed, P_{corr}<0.006) while other VEGF receptor levels were not significantly altered. However, NRP1 can also act as co-receptor with VEGFR2 (Soker et al 1998). Interestingly, one FGF isoform was found significantly differential in some tumor combinations; FGF7 was elevated in colon and prostate xenograft tumors (1.5-fold, P_{corr}<8.7e-6 and 3.7-fold, P_{corr}<7.5e-7) respectively but 2-fold suppressed in ovarian tumors (P_{corr}<0.006), FIG. 5. FGF7 was previously shown to stimulate the growth of endothelial cells of small but not large vessels in the rat cornea (Gillis et al 1999) and hence supports the notion of vascular remodeling versus vasculogenesis. That differential expression of this gene was found only in some tumor combinations is consistent with the concept that each type of tumor will display individual differences in the balance angiogenic activators and inhibitors, yet the end physiological result, increased tumor vascularization, is the same (Bergers et al 2003). Finally, as noted above, genes that help destabilize or remodel vessels such as ANGPT2 and ANGPTL4 were induced, consistent with an overall pattern of late-stage angiogenesis. Interestingly, three genes involved in neuropeptide signaling or peptide binding were found to be significantly differential between xenograft tumors and their parental cell lines: neuropeptide Y receptor Y1 (NPY1R), melanocortin-2 receptor (MC2R), and SORCS3/neurotensin receptor gene. NPY1R is a GPCR that functions as a neuropeptide receptor and was identified by the pooled ANOVA analysis and the linear discriminant analysis. Supporting this observation, previous expression profile studies have found NPY1R to be substantially induced in many breast, prostate and pancreatic carcinomas (Su et al 2001). Both MC2R and the SORCS3 were found to be differentially expressed in the pooled ANOVA analysis. MC2R is a GPCR that binds the ACTH peptide while SORCS3 is a homolog of the rat sortilin gene with VPS10 domains characteristic to neuropeptide-binding proteins (Hampe et al 2001, Lintzel et al 2002, Vincent et al 1999). ACTH has been found to increase angiogenesis of cultured endothelial cells in a 3D-collagen assay (Mallet et al 2003). Similarly, neuropeptide Y has been reported to trigger angiogenesis via the NPY2 receptor in ischemic muscle of mice (Lee et al 2003) and chick endothelial migration assays (Ekstrand et al 2003). Other neuropeptides have been implicated in stimulating VEGF in prostate cancer cells (Levine et al 2003). The neuropeptide Y1 receptor subtype has also been implicated in mediating neuroproliferation (Hansel et al 2001).

Primary human tumors from any single tissue source exhibit diverse and complex expression behavior (Perou et al 1999, Su et al 2001); the strategies described herein could be used to examine several established lines from many histologically similar primary tumors as well as different tumor types from the same tissue. Given the multiple cell types within the tumors, the xenograft model described herein may also be used to analyze micro-dissected xenograft or primary tumors. Additionally, the xenograft model can be more readily extended to monitor time-dependent expression profile changes in the development of tumors. Such results can be used in combination or as a filter with other biomarker technologies such as tissue arrays (Hoos et al 2001) or mass spectroscopy (Petricoin et al 2002) to fuilly characterize clinical specimens for diagnostic or prognostic purposes.

It should be noted that the foregoing description is only illustrative of the invention. Various alternatives and modifications can be devised by those skilled in the art without departing from the invention. Accordingly, the invention is intended to embrace all such alternatives, modifications and variances which fall within the scope of the disclosed invention.

### EXAMPLES

Custom array design. A two-stage strategy was employed to design the custom oligonucleotide microarray chip. First, for the known secreted and cell surface proteins, keyword filtering was performed with respect to the gene descriptions and annotations of curated public databases such as SwissProt/Trembl, the Gene Ontology tables, the UCSC Human Genome assembly (hg13, NCBI Build 31), the GPCR database and public gene tables from technical supply vendors (Affymetrix, Agilent and Illumina). Some of the keywords used were “secreted”, “trans-membrane”, “glycosylated” and “olfactory”. Redundancies and false positives were removed by manual curation.

In order to accommodate continued optimization of a custom chip design, a chip platform was chosen that met several criteria: it must allow rapid changes to the master template even for small production batches, possess relative high density, exhibit strong signal-to-noise properties and have high reproducibility (CV<10%). Hence, a custom oligonucleotide-based microarray chip (Agilent, Palo Alto, Calif.) was designed using the curated collection of secreted and cell surface proteins with human-specific 60-mer probes derived from the 3′ 1500 nt region of each mRNA sequence. The custom chip was designed with a matrix of technical probe replicates and multiple probes for some genes; e.g., 2 or 3 probes with 1, 3 or 5 copies each per array reed some genes. All probes were curated by elimination of sequences with unfavorable Tₘ properties, predicted secondary structure or homo-polymer regions. Finally, Blastn analysis was used to confirm human specificity by comparison to mouse sequences.

Cell lines and mice. All cell lines (A549, MDA MB-231, HCT-116, SK-OV3, and PC3) were obtained from the ATCC (Manassas, Va.). Xenograft tumors were generated from each parental cell line by either implantation of cells or passage of a fragment from a primary tumor (Piedmont Research Center, Morrisville, N.C.). For the A549, MDA MB-231 and SKOV-3 lines, 1×10⁷ cells were implanted subcutaneously into the flank of between 8 and 10 BalbC (Harlan Labs, Indianapolis, Ind.) mice. Between 50 and 75% of the mice yielded a palpable primary xenograft tumor. For the HCT116 and PC3 xenograft tumors, 1 mm³ tumor fragments between 103-110 mg were excised from a primary xenograft tumor and passed into secondary mice for the HCT-116 and PC3 xenograft tumors employed in this study. For PC3 tumors, 8 male mice were implanted with fragments; otherwise recipient mice were female.

RNA preparation. For the parental cell lines, total RNA was harvested from 4×10⁶ cells using a High Pure RNA isolation kit (Roche Applied Science, Indianapolis, Ind.) according to manufacturer's instructions. Tumors were excised 22-29 days post-implantation under accredited procedures (Piedmont Research Center, Morrisville, N.C.), snap-frozen in liquid nitrogen and stored at −80° C. until use. Total RNA was prepared from frozen specimens by 24 hr immersion at −80° C. in RNAlater-ICE (Ambion, Austin, Tex.) to ‘transition’ solid tumors for subsequent homogenization by grinding with a liquid nitrogen-chilled mortar/pestle, followed by resuspension in Trizol (Sigma-Aldrich, E. St. Louis, Mo.) and sonication to complete the tissue disruption. Total RNA was extracted using Phase-lock gels (Brinkmann Brinkmann, Westbury, N.Y.), ethanol precipitated, resuspended in RNase-free water, and aliquoted prior to use. Quality control of the total RNA was facilitated by the use of a microcapillary electrophoresis system (Agilent 2100 Bioanlyzer; Agilent Technologies, Palo Alto, Calif.).

Experimental Design and Array Hybridization. To identify cell surface genes that are consistently differentially regulated amongst the derivative tumors, multiple tumor specimens and their parental source cell lines were hybridized to the custom chips. All biological specimens were co-hybridized with a reference cDNA synthesized from mRNA that is mixture of 10 human established cell lines (Universal RNA; Stratagene, Carlsbad, Calif.). For each array, amino-allyl labeled single-stranded cDNA was synthesized from 10 mg of sample total RNA and from 10 μg universal RNA using the Agilent Fluorescent Direct Label Kit according to manufacturer's instructions, except that a dNTP mix containing 5-[3-Aminoallyl]-2′-deoxyuridine 5′- triphosphate (AA-dUTP; Sigma-Aldrich) was used (final concentration: 100 mM dATP, dCTP, M dGTP; 50 mM dTTP, AA-dUTP). Amino-allyl labeled cDNA was purified using QIAquick PCR M columns (Qiagen, Valencia Calif.) and coupled to either N-hydroxysuccinimidyl-esterified Cy3 or Cy5 dyes (Cy-Dye mono-functional NHS ester; Amersham, Piscataway N.J.). Dye-conjugated cDNAs were purified from free dye using the CyScribe GFX purification kit (Amersham). Targets were hybridized to the microarray for 16 hrs at 60° C. using an Agilent In Situ Hybridization Kit per manufacture's instructions, washed 10 min in 6× SSC, 0.005% Triton X-102 at 22° C., 0.1× SSC, 0.005% Triton X-102 for 10 min at 4° C., dried under a stream of nitrogen, and scanned with an Agilent Microarray Scanner. Hybridization signals were extracted with Agilent Feature Extraction Software version 7.1, which yielded the median of all pixel intensities for each feature. Since two identical arrays of 8500 features were printed on each chip, a complete dye-swap comparison could be performed per chip. For example, on the left array, a Cy3-labeled biological specimen was co-hybridized with Cy5-labeled cDNA made from universal RNA. For the cognate dye-swap experiment on the right array, a Cy-5 labeled biological specimen was co-hybridized with Cy3-labeled cDNA made from universal RNA. Each of these chips was replicated 3 times for each tumor or parental cell line sample. To enable identification of differentially expressed genes with higher statistical reliability, both dye-swap hybridizations and triplicate arrays were routinely performed for each sample.

Quantitative PCR. Real-time (RT-) PCR analysis of selected RNA transcripts was performed using either a GeneAmp 5700 Sequence Detection System or an ABI PRISM 7900HT Sequence Detection System with SyBr green chemistry (Applied Biosystems, Foster City, Calif.). The cDNA produced by reverse transcribing the equivalent of 10 ng of total RNA was loaded per RT-PCR reaction. The following primers pairs were used: beta actin (ACTB) CCTGGCACCCAGCACAAT CCTGGCACCCAGCACAAT (SEQ ID NO:1), GCCGATCCACACGGAGTACT GCCGATCCACACGGAGTACT (SEQ ID NO:2); Human osteopontin (HSPP); AGCAAAATGAAAGAGAACATGAAATG AGCAAAATGAAAGAGAACATGAAATG (SEQ ID NO:3), TTCAACCAATAAACTGAGAAAGAAGC TTCAACCAATAAACTGAGAAAGAAGC (SEQ ID NO:4); murine osteopontin (mSpp); ATTTTGGGCTCTTAGCTTAGTCTGTT ATTTTGGGCTCTTAGCTTAGTCTGTT (SEQ ID NO:5), GGTTACAACGGTGTTTGCATGA GGTTACAACGGTGTTTGCATGA (SEQ ID NO:6); angiopoietin-like 4 (ANGPTL4); ATGTGGCCGTTCCCTGC ATGTGGCCGTTCCCTGC (SEQ ID NO:7), TCTTCTCTGTCCACAAGTTTCCAG TCTTCTCTGTCCACAAGTTTCCAG (SEQ ID NO:8); chemokine (C-C motif) receptor 4 (CCR4); ATTCCTGAGCCAGTGTCAGGAG ATTCCTGAGCCAGTGTCAGGAG (SEQ ID NO:9), CTGTCTTTCCACTGTGGGTGTAAG CTGTCTTTCCACTGTGGGTGTAAG (SEQ ID NO:10); fibroblast growth factor 23 (FGF23); GGCAAAGCCAAAATAGCTCC GGCAAAGCCAAAATAGCTCC (SEQ ID NO:11), CTGCCACATGACGAGGGATAT CTGCCACATGACGAGGGATAT (SEQ ID NO:12); G protein, alpha activating activity polypeptide O (GNAO1) CTAGTCTTTGGGAAACGGGTTGT CTAGTCTTTGGGAAACGGGTTGT (SEQ ID NO:13), AAATCCAACACGGCAAAGGA AAATCCAACACGGCAAAGGA (SEQ ID NO:14); glycoprotein 2; (GP2) GCTTTCCACTCCAATTCACACA GCTTTCCACTCCAATTCACACA (SEQ ID NO:15), CCTGGCCTTGATTCTGTTAATACC CCTGGCCTTGATTCTGTTAATACC (SEQ ID NO:16); collagen, type I, alpha 1; (COL1A1) TCCCCAGCTGTCTTATGGCT TCCCCAGCTGTCTTATGGCT (SEQ ID NO:17), CAGCACGGAAATTCCTCC CAGCACGGAAATTCCTCC (SEQ ID NO:18); G protein-coupled receptor 10; (GPR10) CATGCTCGAGTCATCAGCCA CATGCTCGAGTCATCAGCCA (SEQ ID NO:19), TTTCACTGCCCCCTTTGTGT TTTCACTGCCCCCTTTGTGT (SEQ ID NO:20); G protein-coupled receptor 110; (GPR110) AAGCTCTGGAGGCCGACTG AAGCTCTGGAGGCCGACTG (SEQ ID NO:21), GGCCTTGTCATCCCGACTC GGCCTTGTCATCCCGACTC (SEQ ID NO:22); (CD44); TACAGCATCTCTCGGACGGAG TACAGCATCTCTCGGACGGAG (SEQ ID NO:23), GGTGCTATTGAAAGCCTTGCA GGTGCTATTGAAAGCCTTGCA (SEQ ID NO:24); (CD81); CCCTAAGTGACCCGGACACTT CCCTAAGTGACCCGGACACTT (SEQ ID NO:25), CGTTATATACACAGGCGGTGATG CGTTATATACACAGGCGGTGATG (SEQ ID NO:26). The identity of each amplicon was confirmed by melting curve analysis at the end of the RT-RTPCR run.

Array Analysis. While the array vendor's feature extraction software ‘processed’ the hybridization signal to correct for image intensity, background and minor spatial artifacts, chip- chipto- chip comparisons such as ‘reference’ versus ‘experimental’ sample were handled by a custom to-database (Osborne et al 2003) built upon MySQL with a web interface served by Apache. The database allows the control of experimental design and specification of comparisons and analyses to be performed. Some calculations, like t-tests and ratios, can be performed in the database or its interface layer, but MATLAB (Mathworks, Natick, Mass.) was used for ANOVA and principal components analysis (PCA).

For identification of differentially expressed genes, the MAANOVA package (see The Jackson Laboratory web site) an implementation of ANOVA for microarray analysis (Kerr et al 2001) was used. Array data were loaded into the database and minimally pre-processed for use with this package: where replicate features of the same probe existed in the array design, means were calculated to yield a single expression level for each probe. All signals were Log2 transformed prior to subsequent analyses. These data were used to fit a linear model with factors Gene, Array, Array x Gene, Dye, Dye x Gene, and Sample x Gene. This last attribute is the quantity used for analysis, reing the differential expression of a given gene under a given experimental condition, with the other factors serving to normalize the data. In order to identify differential expression these residuals were analyzed with three statistical tests: a standard ANOVA F-test and two minor variations. A probe had to pass these three tests, generally at 99.9% significance, in order to be called as differentially expressed. A permutation analysis and one-step multiple comparisons correction were applied in conjunction with these tests. It should be noted that since three tests are applied, three P-values result, and when single P-values are listed; the maximum of the three P-values is reported. Finally, because all samples were co-hybridized with cDNAs made from a universal RNA sample, for comparisons of differential gene behavior, approximate ‘ratios’ were calculated by dividing the paired individual tumor/universal RNA ratio by the paired parental cell/universal RNA ratio.

Ontology Annotation. Unigene Gene names were classified by the consistent terms of the Gene Ontology™ consortium and the fatiGO interface to the Gene Ontology.

### REFERENCES

The following references, cited above, are incorporated herein in their entirety by reference:

Aonuma M, Iwahana M, Nakayama Y, Hirotani K, Hattori C, Murakami K, Shibuya M, Tanaka N G. Tumorigenicity depends on angiogenic potential of tumor cells: dominant role of vascular endothelial growth factor and/or fibroblast growth factors produced by tumor cells. Angiogenesis. 1998;2(1):57-66.

Benjamin L E, Hemo I, Keshet E. A plasticity window for blood vessel remodeling is defined by pericyte coverage of the preformed endothelial network and is regulated by PDGF-B and VEGF. Development. 1998 May;125(9):1591-8.

Bergers G, Benjamin L E. Tumorigenesis and the angiogenic switch. Nat Rev Cancer. 2003 June;3(6):401-10.

Cailleau R, Young R, Olive M, Reeves W J Jr. ung R, Olive M, Reeves W J Jr. Breast tumor cell lines from pleural effusions. J Natl Cancer Inst. 1974 September;53(3):661-74.

Carson-Walter E B, Watkins D N, Nanda A, Vogelstein B, Kinzler K W, St Croix B. Cell surface tumor endothelial markers are conserved in mice and humans. Cancer Res. 2001 Sept. 15;61(18):6649-55.

Creighton C, Kuick R, Misek D E, Rickman D S, Brichory F M, Rouillard J M, Omenn G S, Hanash S. Profiling of pathway-specific changes in gene expression following growth of human cancer cell lines transplanted into mice. Genome Biol. 2003;4(7):R46. Epub 2003 Jun. 23.

Dhanasekaran S M, Barrette T R, Ghosh D, Shah R, Varambally S, Kurachi K, Pienta K J, Rubin M A, Chinnaiyan A M. Delineation of prognostic biomarkers in prostate cancer. Nature. 2001 Aug. 23;412(6849):822-6.

Ekstrand A J, Cao R, Bjomdahl M, Nystrom S, Jonsson-Rylander A C, Hassani H, Hallberg B, Nordlander M, Cao Y. Deletion of neuropeptide Y (NPY) 2 receptor in mice results in blockage of NPY-induced angiogenesis and delayed wound healing. Proc Natl Acad Sci U S A. 2003 May 13;100(10):6033-8. Epub 2003 May 02.

Fidler I J The pathogenesis of cancer metastasis: the “seed and soil” hypothesis revisted. Nature Rev Cancer. 2003 Jun;3(6):453-8. Fukuda R, Kelly B, Semenza G L. Vascular endothelial growth factor gene expression in colon cancer cells exposed to prostaglandin E2 is mediated by hypoxia-inducible factor 1. Cancer Res. 2003 May 1;63(9):2330-4.

Fukuoka S, Freedman S D, Scheele G A. Edman S D, Scheele G A. A single gene encodes membrane-bound and free forms of GP-2, the major glycoprotein in pancreatic secretory (zymogen) granule membranes. Proc Natl Acad Sci U S A. 1991 Apr. 1;88(7):2898-902.

Gallo O, Franchi A, Magnelli L, Sardi I, Vannacci A, Boddi V, Chiarugi V, Masini E. Cyclooxygenase-2 pathway correlates with VEGF expression in head and neck cancer. Implications for tumor angiogenesis and metastasis. Neoplasia. 2001 January-February;3(1):53-61.

Gerhardt H, Betsholtz C. Endothelial-pericyte interactions in angiogenesis. Cell Tissue Res. 2003 October;314(1):15-23.

Giard D J, Aaronson S A, Todaro G J, Arnstein P, Kersey J H, Dosik H, Parks W P. In vitro cultivation of human tumors: establishment of cell lines derived from a series of solid tumors. J Natl Cancer Inst. 1973 November;51(5):1417-23.

Gillis P, Savla U, Volpert O V, Jimenez B, Waters C M, Panos R J, Bouck N P. Keratinocyte growth factor induces angiogenesis and protects endothelial barrier function. J Cell Sci. 1999 June;112 ( Pt 12):2049-57.

Glinsky G V, Krones-Herzig A, Glinskii A B, Gebauer G. Microarray analysis of xenograft-derived cancer cell lines reing multiple experimental models of human prostate cancer. Mol Carcinog. 2003 August;37(4):209-21.

Hampe W, Rezgaoui M, Hermans-Borgmeyer I, Schaller H C. The genes for the human VPS10 domain-containing receptors are large and contain many small exons. Hum Genet. 2001 June;108(6):529-36.

Hansel D E, Eipper B A, Ronnett G V. Neuropeptide Y functions as a neuroproliferative factor. Nature. 2001 Apr. 19;410(6831):940-4.

Harada S, Nagy J A, Sullivan K A, Thomas K A, Endo N, Rodan G A, Rodan S B. Induction of vascular endothelial growth factor expression by prostaglandin E2 and E1 in osteoblasts. J Clin Invest. 1994 June;93(6):2490-6.

Hattori K, Dias S, Heissig B, Hackett N R, Lyden D, Tateno M, Hicklin D J, Zhu Z, Witte L, Crystal R G, Moore M A, Rafii S. s S, Heissig B, Hackett N R, Lyden D, Tateno M, Hicklin D J, Zhu Z, Witte L, Crystal R G, Moore M A, Rafii S. Vascular endothelial growth factor and angiopoietin-1 stimulate postnatal hematopoiesis by recruitment of vasc J Exp Med. 2001 May 7;193(9):1005-14.

Hirama M, Takahashi F, Takahashi K, Akutagawa S, Shimizu K, Soma S, Shimanuki Y, Nishio K, Fukuchi Y. Osteopontin overproduced by tumor cells acts as a potent angiogenic factor contributing to tumor growth. Cancer Lett. 2003 Jul. 30;198(1):107-17.

Holash J, Wiegand S J, Yancopoulos G D. New model of tumor angiogenesis: dynamic balance between vessel regression and growth mediated by angiopoietins and VEGF. Oncogene. 1999 Sep. 20;18(38):5356-62.

Hoos A, Urist M J, Stojadinovic A, Mastorides S, Dudas M E, Leung D H, Kuo D, Brennan M F, Lewis J J, Cordon-Cardo C. M J, Stojadinovic A, Mastorides S, Dudas M E, Leung D H, Kuo D, Brennan M F, Lewis J J, Cordon- Cardo C. Validation of tissue microarrays for immunohistochemical profiling of cancer specimens using the example of human fib Am J Pathol. 2001 April;158(4):1245-51.

Kaighn M E, Narayan K S, Ohnuki Y, Lechner J F, Jones L W. Establishment and characterization of a human prostatic carcinoma cell line (PC-3). Invest Urol. 1979 July;17(1):16-23.

Kerr M K, Churchill G A. Experimental design for gene expression microarrays. Biostatistics. 2001 June;2(2):183-201.

Le Jan S, Amy C, Cazes A, Monnot C, Lamande N, Favier J, Philippe J, Sibony M, Gasc J M, Corvol P, Germain S. Angiopoietin-like 4 is a proangiogenic factor produced during ischemia and in conventional renal cell carcinoma. Am J Pathol. 2003 May;162(5):1521-8.

Leali D, Dell Era P, Stabile H, Sennino B, Chambers A F, Naldini A, Sozzani S, Nico B, Ribatti D, Presta M. Osteopontin (Eta-1) and fibroblast growth factor-2 cross-talk in angiogenesis. J Immunol. 2003 Jul. 15;171(2):1085-93.

Lee D K, Nguyen T, Lynch K R, Cheng R, Vanti W B, Arkhitko O, Lewis T, Evans J F, George S R, O'Dowd B F. Discovery and mapping of ten novel G protein-coupled receptor genes. Gene. 2001 Sep. 5;275(1):83-91.

Lee E W, Michalkiewicz M, Kitlinska J, Kalezic I, Switalska H, Yoo P, Sangkharat A, Ji H, Li L, Michalkiewicz T, Ljubisavljevic M, Johansson H, Grant D S, Zukowska Z.

Neuropeptide Y induces ischemic angiogenesis and restores function of ischemic skeletal muscles. J Clin Invest. 2003 June;111(12): 1853-62.

Levine L, Lucci J A 3rd, Pazdrak B, Cheng J Z, Guo Y S, Townsend C M Jr, Hellmich M R. Bombesin stimulates nuclear factor kappa B activation and expression of proangiogenic factors in prostate cancer cells. Cancer Res. 2003 July 1;63(13):3495-502.

Lintzel J, Franke I, Riedel I B, Schaller H C, Hampe W. Characterization of the VPS10 domain of SorLA/LR11 as binding site for the neuropeptide HA. Biol Chem. 2002 November;383(11): 1727-33.

Lopez de Castro J A. HLA-B27 and HLA-A2 subtypes: structure, evolution and function. Immunol Today. 1989 July; 10(7):239-46.

Maeshima Y, Colorado P C, Torre A, Holthaus K A, Grunkemeyer J A, Ericksen M B, Hopfer H, Xiao Y, Stillman I E, Kalluri R. lorado P C, Torre A, Holthaus K A, Grunkemeyer J A, Ericksen M B, Hopfer H, Xiao Y, Stillman I E, Kalluri R. Related Articles, Links Free Full Text Distinct antitumor properties of a type IV collagen domain derived from basement membrane. J Biol Chem. 2000 July 14 14;275(28):21340-8.

Mallet C, Feraud O, Ouengue-Mbele G, Gaillard I, Sappay N, Vittet D, Vilgrain I. Differential expression of VEGF receptors in adrenal atrophy induced by dexamethasone: a protective role of ACTH. Am J Physiol Endocrinol Metab. 2003 January;284(1):E156-67.

Oh Y, Nagalla S R, Yamanaka Y, Kim H S, Wilson E, Rosenfeld R G. Synthesis and characterization of insulin-like growth factor-binding protein (IGFBP)-7. Recombinant human mac25 protein specifically binds IGF-I and -II. J Biol Chem. 1996 Nov. 29;271(48):30322-5.

Paget S. The distribution of secondary growths in cancer of the breast. Lancet, 1889 1:571-3.

Pedersen N, Mortensen S, Sorensen S B, Pedersen M W, Rieneck K, Bovin L F, Poulsen H S. Transcriptional gene expression profiling of small cell lung cancer cells. Cancer Res. 2003 Apr. 15;63(8):1943-53.

Perou C M, Jeffrey S S, van de Rijn M, Rees C A, Eisen M B, Ross D T, Pergamenschikov A, Williams C F, Zhu S X, Lee J C, Lashkari D, Shalon D, Brown P O, Botstein D. Distinctive gene expression patterns in human mammary epithelial cells and breast cancers. Proc Natl Acad Sci U S A. 1999 Aug. 3 3;96(16):9212-7.

Perou C M, Sorlie T, Eisen M B, van de Rijn M, Jeffrey S S, Rees C A, Pollack J R, Ross D T, Johnsen H, Akslen L A, Fluge O, Pergamenschikov A, Williams C, Zhu S X, Lonning P E, Borresen-Dale A L, Brown P O, Botstein D. Molecular portraits of human breast tumours. Nature. 2000 Aug. 17;406(6797):747-52.

Petricoin E F, Ardekani A M, Hitt B A, Levine P J, Fusaro V A, Steinberg S M, Mills G B, Simone C, Fishman D A, Kohn E C, Liotta L A. Use of proteomic patterns in serum to identify ovarian cancer. Lancet. 2002 Feb. 16;359(9306):572-7.

Qi J H, Ebrahem Q, Moore N, Murphy G, Claesson-Welsh L, Bond M, Baker A, Anand-Apte B A novel function for tissue inhibitor of metalloproteinases-3 (TIMP3): inhibition of angiogenesis by blockage of VEGF binding to VEGF receptor-2. Nature Med. 9: 407-415, 2003.

Rittling S R, Chen Y, Feng F, Wu Y. Tumor-derived osteopontin is soluble, not matrix associated. J Biol Chem. 2002 Mar. 15 15;277(11):9175-82. Epub ;2001 Dec. 12.

Roscic-Mrkic B, Fischer M, Leemann C, Manrique A, Gordon C J, Moore J P, Proudfoot A E, Trkola A. RANTES (CCL5) utilizes the proteoglycan CD44 as an auxiliary receptor to mediate cellular activation signals and HIV-1 enhancement. Blood. 2003 Apr. 24.

Ross D T, Scherf U, Eisen M B, Perou C M, Rees C, Spellman P, Iyer V, Jeffrey S S, Van de Rijn M, Waltham M, Pergamenschikov A, Lee J C, Lashkari D, Shalon D, Myers T G,

Weinstein J N, Botstein D, Brown P O. Systematic variation in gene expression patterns in human cancer cell lines. Nat Genet. 2000 March;24(3):227-35.

Salceda S, Macina R A, Turner L R, Sun Y, Liu C., Compositions and methods relating to breast specific genes and proteins. WO 03/106648 (Dec. 24, 2003).

Shirakawa K, Wakasugi H, Heike Y, Watanabe I, Yamada S, Saito K, Konishi F. Vasculogenic mimicry and pseudo-comedo formation in breast cancer. Int J Cancer. 2002 Jun. 20;99(6):821-8.

Soker S, Takashima S, Miao H Q, Neufeld G, Klagsbrun M. Neuropilin-1 is expressed by endothelial and tumor cells as an isoform-specific receptor for vascular endothelial growth factor. Cell. 1998 Mar. 20;92(6):735-45.

Su A I, Welsh J B, Sapinoso L M, Kern S G, Dimitrov P, Lapp H, Schultz P G, Powell S M, Moskaluk C A, Frierson H F Jr, Hampton G M. Molecular classification of human carcinomas by use of gene expression signatures. Cancer Res. 2001 Oct. 15;61(20):7388-93.

Vajkoczy P, Farhadi M, Gaumann A, Heidenreich R, Erber R, Wunder A, Tonn J C, Menger M D, Breier G. Microtumor growth initiates angiogenic sprouting with simultaneous expression of VEGF, VEGF receptor-2, and angiopoietin-2. J Clin Invest. 2002 March;109(6):777-85.

Vincent J P, Mazella J, Kitabgi P. Neurotensin and neurotensin receptors. Trends Pharmacol Sci. 1999 July;20(7):302-9.

Yamauchi T, Umeda F, Masakado M, Isaji M, Mizushima S, Nawata H. Purification and molecular cloning of prostacyclin-stimulating factor from serum-free conditioned medium of human diploid fibroblast cells. Biochem J. 1994 Oct. 15;303 (Pt 2):591-8.

Yoon J C, Chickering T W, Rosen E D, Dussault B, Qin Y, Soukas A, Friedman J M, Holmes, W E, Spiegelman B M Peroxisome proliferator-activated receptor gamrnma target gene encoding a novel angiopoietin-related protein associated with adipose differentiation. Molec. Cell. Biol. 20: 5343-5349, 2000.

Yoshida K, Shimizugawa T, Ono M, Furukawa H. Angiopoietin-like protein 4 is a potent hyperlipidemia-inducing factor in mice and inhibitor of lipoprotein lipase. J Lipid Res. 2002 November;43(11):1770-2.

Zagzag D, Hooper A, Friedlander D R, Chan W, Holash J, Wiegand S J, Yancopoulos G D, Grumet M. In situ expression of angiopoietins in astrocytomas identifies angiopoietin-2 as an early marker of tumor angiogenesis. Exp Neurol. 1999 October October;159(2):391-400.

Zukowska-Grojec Z, Dayao E K, Karwatowska-Prokopczuk E, Hauser G J, Doods H N. Stress-induced mesenteric vasoconstriction in rats is mediated by neuropeptide Y Y1 receptors. Am J Physiol. 1996 February February;270(2 Pt 2):H796-800.

TABLE 1

| Gene Ontology | | |
|---|---|---|
| reference | function | genes |
| GO: 0006928 | cell motility | HAS1 (hyaluronan synthase 1; HAS) |
| | | TSPAN-3 (tetraspan TM4SF; tetraspanin 3; |
| | | tetraspanin TM4-A; transmembrane 4 superfamily |
| | | member 8) |
| | | FN1 (fibronectin 1; cold-insoluble globulin; CIG; |
| | | FINC; FN; LETS) |
| | | IL8 (interleukin 8; CXC chemokine ligand 8; |
| | | LUCT/interleukin-8; T cell chemotactic factor; beta- |
| | | thromboglobulin-like protein; emoctakin; |
| | | granulocyte chemotactic protein 1; lymphocyte- |
| | | derived neutrophil-activating factor; monocyte |
| | | derived neutrophil-activating protein; monocyte- |
| | | derived neutrophil chemotactic factor; neutrophil- |
| | | activating factor; neutrophil-activating peptide 1; |
| | | neutrophil-activating protein 1; protein 3-10C; |
| | | small inducible cytokine subfamily B, member 8; 3- |
| | | 10C; AMCF-I; CXCL8; GCP-1; GCP1; IL-8; K60; |
| | | LECT; LUCT; LYNAP; MDNCF; MONAP; NAF; |
| | | NAP-1; NAP1; SCYB8; TSG-1; b-ENAP) |
| GO: 0006950 | response to stress | CXCL2 (chemokine (C-X-C motif) ligand 2; GRO2 |
| | | oncogene; CINC-2a; GRO2; GROB; GROb; MGSA- |
| | | b; MIP-2a; MIP2; MIP2A; SCYB2) |
| | | CXCL1 (chemokine (C-X-C motif) ligand 1 |
| | | (melanoma growth stimulating activity, alpha); |
| | | GRO1 oncogene (melanoma growth stimulating |
| | | activity, alpha); GRO1 oncogene (melanoma |
| | | growth-stimulating activity); GRO1; GROA; GROa; |
| | | MGSA; MGSA-a; NAP-3; SCYB1) |
| | | SPP1 (secreted phosphoprotein 1 (osteopontin, bone |
| | | sialoprotein I, early T-lymphocyte activation 1); |
| | | Secreted phosphoprotein-1 (osteopontin, bone |
| | | sialoprotein); BNSP; BSPI; ETA-1; OPN) FN1 |
| | | SPP1 |
| | | IL8 |
| GO: 0007154 | cell | MAGP2 (microfibril-associated glycoprotein 2) |
| | communication | LTBP1 (latent transforming growth factor beta |
| | | binding protein 1) |
| | | PTGER3 (prostaglandin E receptor 3 (subtype |
| | | EP3); Prostaglandin E receptor 3, EP3 |
| | | subtype; EP3) |
| | | COL4A1 (collagen, type IV, alpha 1; collagen IV, |
| | | alpha-1 polypeptide; collagen of basement |
| | | membrane, alpha-1 chain) |
| | | COL12A1 (collagen, type XII alpha 1; BA209D8.1; |
| | | DJ234P15.1) |
| | | IGFBP3 (insulin-like growth factor binding protein |
| | | 3; IBP3) |
| | | GPR48 (G protein-coupled receptor 48; G-protein- |
| | | coupled receptor 48; LGR4) |
| | | CXCL2 |
| | | PCDHB9 |
| | | COL5A1 (collagen, type V, alpha 1) |
| | | TNC (tenascin C (hexabrachion); Hexabrachion |
| | | (tenascin); hexabrachion (tenascin C, cytotactin); |
| | | HXB; TN) |
| | | TZD1 |
| | | CD164 (CD164 antigen, sialomucin, Sialomucin |
| | | CD164; MGC-24; MUC-24) |
| | | CHODL (chondrolectin) |
| | | CXCL1 |
| | | HAS 1 |
| | | LAMB3 (laminin, beta 3 (nicein (125 kD); kalinin |
| | | (140 kD), BM600 (125 kD)); BM600-125 kDa; |
| | | LAMNB1; kalinin-140 kDa; nicein-125 kDa) |
| | | GPR57 (G protein-coupled receptor 57) EFNA1 |
| | | (ephrin-A1; eph-related receptor tyrosine kinase |
| | | ligand 1; eph-related receptor tyrosine kinase ligand |
| | | 1 (tumor necrosis factor, alpha-induced protein 4); |
| | | immediate early response protein B61; tumor |
| | | necrosis factor, alpha-induced protein 4; B61; |
| | | ECKLG; EFL1; EPLG1; LERK1; TNFAIP4) |
| | | FN1 |
| | | LAMB1 (laminin, beta 1) |
| | | SPP1 |
| | | GPR23 (G protein-coupled receptor 23; P2Y5- |
| | | LIKE; P2Y9) |
| | | GPR44 (G protein-coupled receptor 44; |
| | | chemoattractant receptor-homologous molecule |
| | | expressed on TH2 cells; CRTH2) |
| | | PRSS11 (protease, serine, 11 (IGF binding); HTRA; |
| | | HTRA1; HtrA; L56; ORF480) |
| | | PRAP2B |
| | | INHBB (inhibin, beta B (activin AB beta |
| | | polypeptide), Inhibin, beta-2; activin AB beta |
| | | polypeptide precursor) |
| | | NPY1R (neuropeptide Y receptor Y1; Neuropeptide |
| | | Y receptor; NPYR) |
| | | ESR1 (estrogen receptor 1; estrogen receptor 1 |
| | | (alpha); ER; ESR; ESRA; Era; NR3A1) |
| | | IL8 |
| | | KITLG (KIT ligand; mast cell growth factor; stem |
| | | cell factor precursor; KITL; KL-1; Kitl; MGF; SCF; |
| | | SF) |
| GO: 0007397 | histogenesis and | KITLG |
| | organogenesis | |
| GO: 0007599 | hemostasis | TFPI2 |
| GO: 0007631 | feeding behavior | NPY1R |
| GO: 0008151 | cell growth | FSTL1 (follistatin-like 1; follistatin-related protein; |
| | and/or | FRP; FSL1) |
| | maintenance | NOV (nephroblastoma overexpressed gene, CCN3, |
| | | IGFBP9; NOVH) |
| | | IGFBP3 |
| | | RBP4 (retinol binding protein 4, plasma; retinol- |
| | | binding protein 4, interstitial; retinol-binding |
| | | protein 4, plasma) |
| | | MGC2376 (potassium channel tetramerisation |
| | | domain containing 14 (KCTD14)) |
| | | CD164 |
| | | CXCL1 |
| | | TSPNA-3 |
| | | SLC11A3 (solute carrier family 11 (proton-coupled |
| | | divalent metal ion transporters), member 3; iron |
| | | regulated gene 1; FERROPORTIN 1; FPN1; Homo |
| | | sapiens solute carrier family 11 (proton-coupled |
| | | divalent metal ion transporters), member 3 |
| | | (SLC11A3), mRNA.; IRON-REGULATED |
| | | TRANSPORTER 1; IREG1; SOLUTE CARRIER |
| | | FAMILY 11, MEMBER 3; SLC11A3; ferroportin 1; |
| | | iron regulated gene 1; solute carrier family 11 |
| | | (proton-coupled divalent metal ion transporters), |
| | | member 3; FPN1; HFE4; IREG1; MTP1; |
| | | NM_014585.1; SLC11A3) |
| | | SLC16A8 (solute carrier 16 (monocarboxylic acid |
| | | transporters), member 8; monocarboxylate |
| | | transporter 3; MCT3) |
| | | PLEC1 (plectin 1, intermediate filament binding |
| | | protein, 500 kD; plectin 1, intermediate filament |
| | | binding protein, 500 kD; EBS1; PCN; PLTN) |
| | | KTN1 (kinectin 1 (kinesin receptor); CG-1 antigen; |
| | | kinesin receptor; CG1; KIAA0004; KNT) |
| | | SPP1 |
| | | COL5A2 (collagen, type V, alpha 2; AB collagen; |
| | | Collagen V, alpha-2 polypeptide; collagen, fetal |
| | | membrane, A polypeptide) PRSS11 (protease, |
| | | serine, 11 (IGF binding); HTRA; HTRA1; HtrA; |
| | | L56; ORF480) INHBB IGFBP7 (insulin-like growth |
| | | factor binding protein 7; FSTL2; IGFBP-7; MAC25; |
| | | PSF) |
| | | ES1 |
| | | IL8 |
| | | KITLG |
| GO: 0008152 | metabolism | PTGER3 |
| | | KLK13 (kallikrein 13; kallikrein-like gene 4; |
| | | DKFZP586J1923; KLK-L4; KLKL4) |
| | | HAS1 |
| | | SEPP1 (selenoprotein P, plasma, 1; SeP) |
| | | TLL1 (tolloid-like 1; TLL) |
| | | PRSS11 |
| | | MMP7 (matrix metalloproteinase 7 (matrilysin, |
| | | uterine); matrin; uterine matrilysin; MMP-7; |
| | | MPSL1; PUMP-1) |
| | | INHBB |
| | | RNASE4 (ribonuclease, RNase A family, 4; RNS4) |
| | | ESR1 |
| GO: 0009605 | response to | RBP4 |
| | external stimulus | CXCL2 |
| | | CD164 |
| | | CXCL1 |
| | | SEPP1 |
| | | FN1 |
| | | SPP1 |
| | | GPR44 |
| | | INHBB IL8 |
| GO: 0009653 | morphogenesis | ANGPTL4 (angiopoietin-like 4; Alternate Names: |
| | | PPARG angiopoietin related protein; fasting- |
| | | induced adipose factor; hepatic angiopoietin-related |
| | | protein; hepatic fibrinogen/angiopoietin-related |
| | | protein; ANGPTL2; ARP4; FIAF; HFARP; PGAR; |
| | | PP1158; PPARG; pp1158) |
| | | COL12A1 |
| | | PCDHB9 |
| | | CXCL1 |
| | | LAMB3 |
| | | TSPAN3 |
| | | SPP1 |
| | | COL1A1 (collagen, type I, alpha 1; Alternate |
| | | Names: Collagen I, alpha-1 polypeptide; collagen of |
| | | skin, tendon and bone, alpha-1 chain; osteogenesis |
| | | imperfecta type IV; OI4) |
| | | TLL1 |
| | | INHBB |
| | | IL8 |
| GO: 0009791 | post-embryonic | INHBB |
| | development | |
| GO: 0016265 | death | PTGER3 |
| | | SPP1 |
| GO: 0019058 | viral infectious | IL8 |
| | cycle | |
| GO: 0030154 | cell | SPP1 |
| | differentiation | INHBB |
| GO: 0042698 | menstrual cycle | INHBB |
| GO: 0046849 | bone remodeling | SPP1 |
| GO: 0046903 | secretion | INHBB |
| NA | not known | CD63 (CD63 antigen (melanoma 1 antigen); |
| | | granulophysin; lysosome-associated membrane |
| | | glycoprotein 3; melanoma 1 antigen; melanoma- |
| | | associated antigen ME491; melanoma-associated |
| | | antigen MLA1; ocular melanoma-associated |
| | | antigen; LAMP-3; ME491; MLA1; OMA81H) |
| | | FLJ20559 (chromosome 9 open reading frame 95 |
| | | (C9orf95), NRK1, FLJ20559, bA235O14.2) |
| | | GP2 (glycoprotein 2 (zymogen granule membrane); |
| | | pancreatic zymogen granule membrane associated |
| | | protein GP2 beta form; ZAP75) |
| | | AB065858 (seven transmembrane helix receptor) |

TABLE 2

| Ap | Ld | Ai | Gene | Pval | R |
|---|---|---|---|---|---|
| 1 | 1 | 1 | LAMB3 | 0.001 | 1.9 |
| 1 | 1 | 1 | ANGPTL4 | 0.001 | 2.1 |
| 1 | 1 | 1 | COL1A1 | 0.001 | 3.6 |
| 1 | 1 | 1 | PCDHB9 (protocadherin-beta 9, PCDH- | 0.001 | 4.0 |
| | | | BETA-9) | | |
| 1 | 1 | 1 | GPR57 | 0.001 | 5.7 |
| 1 | 1 | 1 | GP2 | 0.001 | 5.7 |
| 1 | 1 | 1 | PTGER3 | 0.001 | 6.4 |
| 1 | 1 | 0 | KITLG | 0.001 | 0.4 |
| 1 | 1 | 0 | RAP2B | 0.001 | 0.4 |
| 1 | 1 | 0 | COL5A1 | 0.237 | 1.0 |
| 1 | 1 | 0 | SEPP1 | 0.054 | 1.0 |
| 1 | 1 | 0 | CXCL1 | 0.3 | 1.1 |
| 1 | 1 | 0 | TNC | 0.001 | 1.3 |
| 1 | 1 | 0 | LTBP1 | 0.009 | 1.3 |
| 1 | 1 | 0 | PRSS11 | 0.001 | 1.3 |
| 1 | 1 | 0 | FN1 | 0.008 | 1.4 |
| 1 | 1 | 0 | FZD1 (frizzled homolog 1 (Drosophila); | 0.019 | 1.4 |
| | | | Frizzled, Drosophila, homolog of, 1; Wnt | | |
| | | | receptor) | | |
| 1 | 1 | 0 | SPP1 | 1 | 1.5 |
| 1 | 1 | 0 | IGFBP7 | 0.008 | 1.7 |
| 1 | 1 | 0 | RNASE4 | 0.008 | 1.9 |
| 1 | 1 | 0 | CHODL | 0.003 | 2.1 |
| 1 | 1 | 0 | NOV | 0.003 | 2.2 |
| 1 | 1 | 0 | COL12A1 | 0.001 | 2.2 |
| 1 | 1 | 0 | MAGP2 | 0.001 | 2.6 |
| 1 | 1 | 0 | GPR23 | 0.574 | 3.0 |
| 1 | 1 | 0 | TLL1 | 0.001 | 3.2 |
| 1 | 1 | 0 | GPR44 | 0.069 | 3.6 |
| 1 | 1 | 0 | MGC2376 | 0.001 | 4.7 |
| 1 | 1 | 0 | NPY1R | 0.183 | 5.2 |
| 1 | 0 | 1 | EMP3 (epithelial membrane protein 3) | 0.004 | 0.5 |
| 1 | 0 | 1 | HLA-A (major histocompatibility complex, | 0.001 | 0.6 |
| | | | class II, DO alpha; HLA-D0-alpha; lymphocyte | | |
| | | | antigen; major histocompatibility complex, | | |
| | | | class II, DN alpha; HLA-D0-alpha; HLA- | | |
| | | | DNA; HLA-DZA; HLADZ) | | |
| 1 | 0 | 1 | GNAO1 (guanine nucleotide binding protein | 0.001 | 2.5 |
| | | | (G protein), alpha activating activity | | |
| | | | polypeptide O; G-ALPHA-o; GNAO) | | |
| 1 | 0 | 0 | CCR5 (chemokine (C-C motif) receptor 5; | 0.001 | 0.2 |
| | | | chemokine (C-C) receptor 5; chemr13; CC- | | |
| | | | CKR-5; CCCKR5; CKR-5; CKR5; CMKBR5) | | |
| 1 | 0 | 0 | C20orf52 (chromosome 20 open reading frame | 0.001 | 0.4 |
| | | | 52; homolog of mouse RIKEN 2010100O12 | | |
| | | | gene; bA353C18.2) | | |
| 1 | 0 | 0 | SORCS3 (VPS10 domain receptor protein; | 0.001 | 0.4 |
| | | | KIAA1059, SORCS) | | |
| 1 | 0 | 0 | PF4 (platelet factor 4; platelet factor 4; | 0.005 | 0.4 |
| | | | CXCL4; SCYB4) | | |
| 1 | 0 | 0 | SPINK2 (serine protease inhibitor, Kazal type, | 0.001 | 0.4 |
| | | | 2 (acrosin-trypsin inhibitor); HUSI-II) | | |
| 1 | 0 | 0 | IGSF6 (immunoglobulin superfamily, member | 0.008 | 0.4 |
| | | | 6) | | |
| 1 | 0 | 0 | GPR110 (G protein-coupled receptor 110; G- | 0.001 | 0.5 |
| | | | protein coupled receptor 110; hGPCR36) | | |
| 1 | 0 | 0 | OR1J5 (olfactory receptor, family 1, subfamily | 0.001 | 0.5 |
| | | | J, member 5; HSA5) | | |
| 1 | 0 | 0 | BGLAP (bone gamma-carboxyglutamate (gla) | 0.001 | 0.5 |
| | | | protein (osteocalcin); Bone gamma- | | |
| | | | carboxyglutamic acid protein; osteocalcin; | | |
| | | | BGP) | | |
| 1 | 0 | 0 | GALR2 (galanin receptor 2; GALNR2) | 0.001 | 0.5 |
| 1 | 0 | 0 | HCN2 (hyperpolarization activated cyclic | 0.001 | 0.5 |
| | | | nucleotide-gated potassium channel 2; brain | | |
| | | | cyclic nucleotide gated channel 2; BCNG-2; | | |
| | | | BCNG2; HAC-1) | | |
| 1 | 0 | 0 | CD81 (CD81 antigen (target of | 0.001 | 0.5 |
| | | | antiproliferative antibody 1); 26 kDa cell | | |
| | | | surface protein TAPA-1; target of | | |
| | | | antiproliferative antibody 1; S5.7; TAPA-1; | | |
| | | | TAPA1) | | |
| 1 | 0 | 0 | OGFR (opioid growth factor receptor; 7-60 | 0.001 | 0.5 |
| | | | protein; zeta-type opioid receptor; 7-60; Jul-60) | | |
| 1 | 0 | 0 | GPR6 (G protein-coupled receptor 6) | 0.001 | 0.5 |
| 1 | 0 | 0 | OMP (olfactory marker protein; Olfactory | 0.001 | 0.5 |
| | | | marker protein) | | |
| 1 | 0 | 0 | CMA1 (chymase 1, mast cell; chymase, heart; | 0.001 | 0.5 |
| | | | chymase, mast cell; mast cell protease I; CYH; | | |
| | | | MCT1) | | |
| 1 | 0 | 0 | DKFZP564D0 | 0.001 | 0.6 |
| 1 | 0 | 0 | CHRM1 (cholinergic receptor, muscarinic 1; | 0.001 | 0.6 |
| | | | muscarinic acetylcholine receptor M1; HM1; | | |
| | | | M1) | | |
| 1 | 0 | 0 | PYY (peptide YY) | 0.001 | 0.6 |
| 1 | 0 | 0 | FGF19 (fibroblast growth factor 19) | 0.004 | 0.6 |
| 1 | 0 | 0 | AGTR2 (angiotensin II receptor, type 2; | 0.047 | 0.6 |
| | | | angiotensin receptor 2; AT2) | | |
| 1 | 0 | 0 | SSTR3 (somatostatin receptor 3) | 0.001 | 0.6 |
| 1 | 0 | 0 | TMPO (thymopoietin; LAP2; TP) | 0.001 | 0.6 |
| 1 | 0 | 0 | TAS2R16 (taste receptor, type 2, member 16; | 0.003 | 0.6 |
| | | | candidate taste receptor T2R16; T2R16) | | |
| 1 | 0 | 0 | ADORA2B (adenosine A2b receptor; | 0.003 | 0.6 |
| | | | ADORA2) | | |
| 1 | 0 | 0 | GPR10 (G protein-coupled receptor 10; | 0.001 | 0.6 |
| | | | prolactin releasing peptide receptor; prolactin- | | |
| | | | releasing hormone receptor; GR3; PrRPR) | | |
| 1 | 0 | 0 | ADCYAP1R1 (adenylate cyclase activating | 0.001 | 0.6 |
| | | | polypeptide 1 (pituitary) receptor type I; | | |
| | | | adenylate cyclase activating polypeptide 1 | | |
| | | | (pituitary) receptor type 1; PACAPR; | | |
| | | | PACAPRI) | | |
| 1 | 0 | 0 | OR1F10 (olfactory receptor, family 1, | 0.001 | 0.6 |
| | | | subfamily F, member 10; OR3-145) | | |
| 1 | 0 | 0 | HDGF (hepatoma-derived growth factor (high- | 0.001 | 0.6 |
| | | | mobility group protein 1-like); Hepatoma- | | |
| | | | derived growth factor; HMG1L2) | | |
| 1 | 0 | 0 | CD151 (CD151 antigen; hemidesmosomal | 0.001 | 0.6 |
| | | | tetraspanin CD151; membrane glycoprotein | | |
| | | | SFA-1; platelet surface glycoprotein gp27; | | |
| | | | platelet-endothelial cell tetraspan antigen 3; | | |
| | | | GP27; PETA-3; SFA-1; SFA1) | | |
| 1 | 0 | 0 | PDAP1 (PDGFA associated protein 1; PDGF | 0.001 | 0.7 |
| | | | associated protein; HASPP28; PAP; PAP1) | | |
| 1 | 0 | 0 | A1BG (alpha-1-B glycoprotein; A1B; ABG; | 0.001 | 0.7 |
| | | | GAB) | | |
| 1 | 0 | 0 | LIPF (lipase, gastric; HGL; HLAL) | 0.001 | 0.7 |
| 1 | 0 | 0 | PBEF (pre-B-cell colony-enhancing factor) | 0.001 | 0.7 |
| 1 | 0 | 0 | ART-4 (Adenocarcinoma antigen recognized | 0.034 | 0.7 |
| | | | by T lymphocytes-4) | | |
| 1 | 0 | 0 | C1QTNF3 (C1q and tumor necrosis factor | 0.029 | 0.7 |
| | | | related protein 3; collagenous repeat-containing | | |
| | | | sequence of 26-kDa; complement-c1q tumor | | |
| | | | necrosis factor-related protein 3; CORS26; | | |
| | | | CTRP3; FLJ37576) | | |
| 1 | 0 | 0 | SLC39A4 (solute carrier family 39 (zinc | 0.022 | 0.7 |
| | | | transporter), member 4; FLJ20327; ZIP4) | | |
| 1 | 0 | 0 | IFNGR2 (interferon gamma receptor 2 | 0.001 | 0.8 |
| | | | (interferon gamma transducer 1); interferon | | |
| | | | gamma receptor accessory factor-1; interferon- | | |
| | | | gamma receptor beta chain precursor; AF-1; | | |
| | | | IFGR2; IFNGT1) | | |
| 1 | 0 | 0 | ENT3 (solute carrier family 29 (nucleoside | 0.001 | 0.8 |
| | | | transporters), member 3 (SLC29A3); | | |
| | | | FLJ11160) | | |
| 1 | 0 | 0 | SERPINC1 (serine (or cysteine) proteinase | 0.001 | 0.8 |
| | | | inhibitor, clade C (antithrombin), member 1; | | |
| | | | antithrombin III; AT3; ATIII; antithrombin III) | | |
| 1 | 0 | 0 | NRP1 (neuropilin 1; NRP; VEGF165R) | 0.006 | 0.8 |
| 1 | 0 | 0 | CACNA1H (calcium channel, voltage- | 0.011 | 0.8 |
| | | | dependent, alpha 1H subunit; calcium channel, | | |
| | | | voltage-dependent, T type, alpha 1Hb subunit; | | |
| | | | CACNA1HB) | | |
| 1 | 0 | 0 | CD44 (CD44 antigen (homing function and | 0.001 | 0.8 |
| | | | Indian blood group system); CD44 antigen | | |
| | | | (homing function); CD44R; IN; MC56; | | |
| | | | MDU2; MDU3; MIC4; Pgp1) | | |
| 1 | 0 | 0 | STC2 (stanniocalcin 2; stanniocalcin related | 0.018 | 0.8 |
| | | | protein; stanniocalcin 2; stanniocalcin related | | |
| | | | protein; STC-2; STCRP) | | |
| 1 | 0 | 0 | DLK1 (delta-like 1 homolog (Drosophila); | 0.064 | 0.8 |
| | | | FA1; PG2; PREF-1; PREF1; Pref-1; ZOG; | | |
| | | | pG2) | | |
| 1 | 0 | 0 | F2R (coagulation factor II (thrombin) receptor; | 0.388 | 0.8 |
| | | | protease-activated receptor 1; thrombin | | |
| | | | receptor; CF2R; PAR1; TR) | | |
| 1 | 0 | 0 | EMP2 (epithelial membrane protein 2; XMP) | 0.001 | 0.8 |
| 1 | 0 | 0 | HBE1 (hemoglobin, epsilon 1) | 0.003 | 0.8 |
| 1 | 0 | 0 | BSG (basigin (OK blood group); M6 antigen; | 0.003 | 0.8 |
| | | | OK blood group; collagenase stimulatory | | |
| | | | factor; emmprin; extracellular matrix | | |
| | | | metalloproteinase inducer; 5F7; CD147; | | |
| | | | EMMPRIN; HGNC: 8130; M6; OK; TCSF) | | |
| 1 | 0 | 0 | GPR80 (G protein-coupled receptor 80; G | 0.001 | 0.8 |
| | | | protein-coupled receptor 99; P2Y-like | | |
| | | | nucleotide receptor; GPR99; HGNC: 14591) | | |
| 1 | 0 | 0 | APOB48R (macrophage receptor for | 0.016 | 0.8 |
| | | | apolipoprotein B48) | | |
| 1 | 0 | 0 | AMELY (amelogenin (Y chromosome); | 0.001 | 0.8 |
| | | | AMGL; AMGY) | | |
| 1 | 0 | 0 | IL26 (interleukin 26; AK155 protein (AK155 | 0.006 | 0.8 |
| | | | gene); AK155; IL-26) | | |
| 1 | 0 | 0 | TRPM5 (transient receptor potential cation | 0.001 | 0.8 |
| | | | channel, subfamily M, member 5; MLSN1 and | | |
| | | | TRP-related; MLSN1- and TRP-related; | | |
| | | | LTRPC5; MTR1) | | |
| 1 | 0 | 0 | ENSA (endosulfine alpha; alpha endosulfine) | 0.001 | 0.8 |
| 1 | 0 | 0 | OR1F1 (olfactory receptor, family 1, subfamily | 0.001 | 0.8 |
| | | | F, member 1; Olfmf; olfactory receptor, family | | |
| | | | 1, subfamily F, member 4; olfactory receptor, | | |
| | | | family 1, subfamily F, member 5; olfactory | | |
| | | | receptor, family 1, subfamily F, member 6; | | |
| | | | olfactory receptor, family 1, subfamily F, | | |
| | | | member 7; olfactory receptor, family 1, | | |
| | | | subfamily F, member 8; olfactory receptor, | | |
| | | | family 1, subfamily F, member 9; | | |
| | | | HGNC: 8198; HGNC: 8199; HGNC: | | |
| | | | 8200; HGNC: 8201; HGNC: 8202; | | |
| | | | HGNC: 8203; OLFMF; OR16-36; | | |
| | | | OR16-37; OR16-88; OR16-89; | | |
| | | | OR16-90; OR1F4; OR1F5; | | |
| | | | OR1F6; OR1F7; OR1F8; | | |
| | | | OR1F9; Olfmf) | | |
| 1 | 0 | 0 | GP3ST (betaGal-3-O-sulfotransferase) | 0.001 | 0.8 |
| 1 | 0 | 0 | BDNF (brain-derived neurotrophic factor; | 0.001 | 0.9 |
| | | | MGC34632) | | |
| 1 | 0 | 0 | PLXN3 (plexin A3; 6.3; Sex chromosome X | 0.005 | 0.9 |
| | | | transmembrane protein of HGF receptor family | | |
| | | | 3; plexin 4; 6.3; PLEXIN-A3; PLXN3; | | |
| | | | PLXN4; Plxn3; SEX; XAP-6) | | |
| 1 | 0 | 0 | ALPMCF1 (APMCF1 protein (non-HGNC | 0.134 | 0.9 |
| | | | gene) | | |
| 1 | 0 | 0 | SCAMP1 (secretory carrier membrane protein | 0.001 | 0.9 |
| | | | 1; SCAMP; SCAMP37) | | |
| 1 | 0 | 0 | PALMD (palmdelphin; chromosome 1 open | 0.001 | 0.9 |
| | | | reading frame 11; paralemnin-like; C1orf11; | | |
| | | | FLJ20271; HGNC: 1231; PALML) | | |
| 1 | 0 | 0 | MMP8 (matrix metalloproteinase 8 (neutrophil | 0.02 | 0.9 |
| | | | collagenase); PMNL collagenase; neutrophil | | |
| | | | collagenase; CLG1; HNC; PMNL-CL) | | |
| 1 | 0 | 0 | MFAP3 (microfibrillar-associated protein 3) | 0.004 | 0.9 |
| 1 | 0 | 0 | SPAG11 (sperm associated antigen 11; | 0.001 | 0.9 |
| | | | epididymal protein 2; sperm associated antigen | | |
| | | | 11 precursor; EP2; EP2C; EP2D; HE2) | | |
| 1 | 0 | 0 | A2M (alpha-2-macroglobulin) | 0.031 | 0.9 |
| 1 | 0 | 0 | NET-2 (transmembrane 4 superfamily member | 0.092 | 0.9 |
| | | | 12; tetraspan NET-2) | | |
| 1 | 0 | 0 | CXCL11 (chemokine (C-X-C motif) ligand 11; | 0.001 | 1.0 |
| | | | small inducible cytokine subfamily B (Cys-X- | | |
| | | | Cys), member 11; small inducible cytokine | | |
| | | | subfamily B (Cys-X-Cys), member 9B; B-R1; | | |
| | | | H174; I-TAC; IP-9; IP9; SCYB11; SCYB9B; | | |
| | | | b-R1) | | |
| 1 | 0 | 0 | KLRB1 (killer cell lectin-like receptor | 0.003 | 1.0 |
| | | | subfamily B, member 1; hNKR-P1A; CD161; | | |
| | | | NKR; NKR-P1; NKR-P1A; NKRP1A; hNKR- | | |
| | | | P1A) | | |
| 1 | 0 | 0 | TF (transferrin; PRO1557) | 0.988 | 1.0 |
| 1 | 0 | 0 | COL14A1 (collagen, type XIV, alpha 1 | 0.001 | 1.0 |
| | | | (undulin); collagen, type XIV, alpha 1; | | |
| | | | undulin; undulin (fibronectin-tenascin-related); | | |
| | | | UND) | | |
| 1 | 0 | 0 | IL7 (interleukin 7; IL-7) | 0.002 | 1.1 |
| 1 | 0 | 0 | COL9A1 (collagen, type IX, alpha 1; cartilage- | 0.001 | 1.1 |
| | | | specific short collagen; collagen IX, alpha-1 | | |
| | | | polypeptide; DJ149L1.1.2; MED) | | |
| 1 | 0 | 0 | CCR4 (chemokine (C-C motif) receptor 4; | 0.001 | 1.1 |
| | | | chemokine (C-C) receptor 4; CC-CKR-4; | | |
| | | | CKR4; CMKBR4; ChemR13; HGCN: 14099; | | |
| | | | K5-5; k5-5) | | |
| 1 | 0 | 0 | FPR1 (formyl peptide receptor 1; FMLP; FPR) | 0.034 | 1.1 |
| 1 | 0 | 0 | FAP (fibroblast activation protein, alpha; | 0.001 | 1.2 |
| | | | integral membrane serine protease; seprase; | | |
| | | | DPPIV; FAPA; SEPRASE) | | |
| 1 | 0 | 0 | OPCML (opioid binding protein/cell adhesion | 0.001 | 1.2 |
| | | | molecule-like; opiate binding-cell adhesion | | |
| | | | molecule; opioid-binding protein/cell adhesion | | |
| | | | molecule-like; OBCAM; OPCM) | | |
| 1 | 0 | 0 | GPR145 (Melanin-concentrating hormone | 0.001 | 1.2 |
| | | | receptor 2; MCH receptor 2; MCHR-2; MCH- | | |
| | | | R2; MCH2R; MCH-2R; MCH2; G protein | | |
| | | | coupled receptor 145) | | |
| 1 | 0 | 0 | GFRA3 (GDNF family receptor alpha 3; | 0.001 | 1.2 |
| | | | GFRalpha3; GPI-linked receptor; glial cell | | |
| | | | line-derived neurotrophic factor receptor alpha- | | |
| | | | 3; GFRA-3; GFRa-3) | | |
| 1 | 0 | 0 | EDN3 (endothelin 3; ET3) | 0.001 | 1.2 |
| 1 | 0 | 0 | IL12B (interleukin 12B (natural killer cell | 0.043 | 1.3 |
| | | | stimulatory factor 2, cytotoxic lymphocyte | | |
| | | | maturation factor 2, p40); IL12, subunit p40; | | |
| | | | IL23, subuint p40; cytotoxic lymphocyte | | |
| | | | maturation factor 2, p40; interkeukin-12 beta | | |
| | | | chain; interleukin 12, p40; interleukin 12B; | | |
| | | | interleukin-12 beta chain; natural killer cell | | |
| | | | stimulatory factor, 40 kD subunit; natural killer | | |
| | | | cell stimulatory factor-2; CLMF; CLMF2; IL- | | |
| | | | 12B; NKSF; NKSF2) | | |
| 1 | 0 | 0 | CXCR4 (chemokine (C-X-C motif), receptor 4 | 0.026 | 1.3 |
| | | | (fusin); Neuropeptide Y receptor Y3; | | |
| | | | chemokine (C-X-C motif), receptor 4 (fusin); | | |
| | | | D2S201E; HM89; HSY3RR; LAP3; LESTR; | | |
| | | | NPY3R; NPYR; NPYY3R; WHIM; fusin) | | |
| 1 | 0 | 0 | PCSK5 (proprotein convertase subtilisin/kexin | 0.427 | 1.3 |
| | | | type 5; prohormone convertase 5; proprotein | | |
| | | | convertase PC5; protease PC6; subtilisin/kexin- | | |
| | | | like protease PC5; PC5; PC6; PC6A; SPC6) | | |
| 1 | 0 | 0 | NID2 (nidogen 2; nidogen 2; nidogen 2 | 0.168 | 1.3 |
| | | | (osteonidogen) | | |
| 1 | 0 | 0 | ITGA4 (integrin, alpha 4 (antigen CD49D, | 0.73 | 1.3 |
| | | | alpha 4 subunit of VLA-4 receptor); antigen | | |
| | | | CD49D, alpha-4 subunit of VLA-4 receptor; | | |
| | | | CD49D; CD49d) | | |
| 1 | 0 | 0 | KIAA1870 (unidentified protein from brain) | 0.016 | 1.3 |
| 1 | 0 | 0 | FBLN5 (fibulin 5; developmental arteries and | 0.001 | 1.4 |
| | | | neural crest epidermal growth factor-like; urine | | |
| | | | p50 protein; DANCE; EVEC; UP50) | | |
| 1 | 0 | 0 | TRPV2 (transient receptor potential cation | 0.001 | 1.4 |
| | | | channel, subfamily V, member 2; vanilloid | | |
| | | | receptor-like protein 1; MGC12549; VRL; | | |
| | | | VRL-1; VRL1) | | |
| 1 | 0 | 0 | FGF23 (fibroblast growth factor 23; | 0.119 | 1.4 |
| | | | Hypophosphatemia vitamin D-resistant rickets- | | |
| | | | 2 (autosomal dominant); tumor-derived | | |
| | | | hypophophatemia inducing factor; ADHR; | | |
| | | | HPDR2; HYPF) | | |
| 1 | 0 | 0 | TEM5 (tumor endothelial marker 5 precursor) | 0.001 | 1.4 |
| 1 | 0 | 0 | CR1 (complement component (3b/4b) receptor | 0.008 | 1.4 |
| | | | 1, including Knops blood group system; C3- | | |
| | | | binding protein; CD35 antigen; complement | | |
| | | | component (3b/4b) receptor-1; C3BR; CD35) | | |
| 1 | 0 | 0 | GPA33 (glycoprotein A33 (transmembrane); | 0.001 | 1.4 |
| | | | A33) | | |
| 1 | 0 | 0 | CLCA4 (chloride channel, calcium activated, | 0.001 | 1.4 |
| | | | family member 4; CACC2; CaCC; CaCC2) | | |
| 1 | 0 | 0 | TIMP3 (tissue inhibitor of metalloproteinase 3 | 0.006 | 1.4 |
| | | | (Sorsby fundus dystrophy, | | |
| | | | pseudoinflammatory); K222 expressed in | | |
| | | | degenerative retinas; Tissue inhibitor of | | |
| | | | metalloproteinase-3; HSMRK222; K222TA2; | | |
| | | | SFD) | | |
| 1 | 0 | 0 | MMP10 (matrix metalloproteinase 10 | 0.001 | 1.4 |
| | | | (stromelysin 2); stromelysin 2; transin 2; SL-2; | | |
| | | | STMY2) | | |
| 1 | 0 | 0 | FUT8 (fucosyltransferase 8 (alpha (1,6) | 0.197 | 1.4 |
| | | | fucosyltransferase); GDP-L-Fuc:N-acetyl-beta- | | |
| | | | D-glucosaminide alpha1,6-fucosyltransferase; | | |
| | | | GDP-fucose--glycoprotein fucosyltransferase; | | |
| | | | alpha1-6FucT; glycoprotein 6-alpha-L- | | |
| | | | fucosyltransferase; MGC26465) | | |
| 1 | 0 | 0 | V1RL1 (putative pheromone receptor V1RL1 | 0.001 | 1.4 |
| | | | long form) | | |
| 1 | 0 | 0 | TRPM5 (transient receptor potential cation | 0.001 | 1.5 |
| | | | channel, subfamily M, member 5; MLSN1 and | | |
| | | | TRP-related; MLSN1-and TRP-related; | | |
| | | | LTRPC5; MTR1) | | |
| 1 | 0 | 0 | EBI2 (Epstein-Barr virus induced gene 2 | 0.003 | 1.5 |
| | | | (lymphocyte-specific G protein-coupled | | |
| | | | receptor); Epstein-Barr virus induced gene | | |
| | | | 2)ADAM28 (a disintegrin and | | |
| | | | metalloproteinase domain 28; ADAM23; | | |
| | | | EMDCII; MDC-LM; MDC-LS; MDC-Lm; | | |
| | | | MDC-Ls; MDCL; eMDCII) | | |
| 1 | 0 | 0 | GPLD1 (glycosylphosphatidylinositol specific | 0.001 | 1.5 |
| | | | phospholipase D1; GPI-specific phospholipase | | |
| | | | D; glycoprotein phospholipase D; | | |
| | | | glycosylphosphatIdylinositol-specific | | |
| | | | phospholipase D; phospholipase D, | | |
| | | | phosphatidylinositol-glycan-specific; GPIPLD; | | |
| | | | GPIPLDM; MGC22590; PIGPLD; PIGPLD1) | | |
| 1 | 0 | 0 | CP (ceruloplasmin (ferroxidase); | 0.008 | 1.5 |
| | | | Ceruloplasmin) | | |
| 1 | 0 | 0 | EPHA3 (EphA3; Ephrin receptor EphA3 | 0.003 | 1.5 |
| | | | (human embryo kinase 1); eph-like tyrosine | | |
| | | | kinase 1; eph-like tyrosine kinase 1 (human | | |
| | | | embryo kinase 1); ephrin receptor EphA3; | | |
| | | | human embryo kinase 1; ETK; ETK1; HEK; | | |
| | | | HEK4; TYRO4) | | |
| 1 | 0 | 0 | KLK11 (kallikrein 11; hippostasin; protease, | 0.003 | 1.5 |
| | | | serine, 20 trypsin-like; protease, serine, trypsin- | | |
| | | | like; MGC33060; PRSS20; TLSP) | | |
| 1 | 0 | 0 | OR7A17 (olfactory receptor, family 7, | 0.001 | 1.6 |
| | | | subfamily A, member 17; HTPCRX19) | | |
| 1 | 0 | 0 | IFI27 (interferon, alpha-inducible protein 27; | 0.001 | 1.6 |
| | | | P27) | | |
| 1 | 0 | 0 | RNASE6 (ribonuclease, RNase A family, k6; | 0.001 | 1.7 |
| | | | RNASEK6; RNS6; RNase; RNase k6; | | |
| | | | RNasek6; k6) | | |
| 1 | 0 | 0 | SELPLG (selectin P ligand; CD162; PSGL-1; | 0.003 | 1.7 |
| | | | PSGL1) | | |
| 1 | 0 | 0 | CST7 (cystatin F (leukocystatin); cystatin 7; | 0.001 | 1.7 |
| | | | cystatin-like metastasis-associated protein; | | |
| | | | leukocystatin; CMAP) | | |
| 1 | 0 | 0 | LEC3 (latrophilin 3 (LPHN3); KIAA0768) | 0.092 | 1.7 |
| 1 | 0 | 0 | TSHR (thyroid stimulating hormone receptor; | 0.001 | 1.7 |
| | | | thyrotropin receptor) | | |
| 1 | 0 | 0 | MC2R (melanocortin 2 receptor | 0.001 | 2.1 |
| | | | (adrenocorticotropic hormone); Melanocortin-2 | | |
| | | | receptor (ACTH receptor); melanocortin 2 | | |
| | | | receptor (adrenocorticotropic hormone | | |
| | | | receptor); ACTHR) | | |
| 1 | 0 | 0 | SV2 (synaptic vesicle glycoprotein 2A; | 0.001 | 2.1 |
| | | | synaptic vesicle glycoprotein 2; KIAA0736; | | |
| | | | SV2) | | |
| 1 | 0 | 0 | SERPINA4 (serine (or cysteine) proteinase | 0.001 | 2.1 |
| | | | inhibitor, clade A (alpha-1 antiproteinase, | | |
| | | | antitrypsin), member 4; protease inhibitor 4 | | |
| | | | (kallistatin); KAL; KLST; KST; PI4; | | |
| | | | kallistatin) | | |
| 1 | 0 | 0 | EBI2 (Epstein-Barr virus induced gene 2 | 0.001 | 2.1 |
| | | | (lymphocyte-specific G protein-coupled | | |
| | | | receptor); Epstein-Barr virus induced gene | | |
| | | | 2)ADAM28 (a disintegrin and | | |
| | | | metalloproteinase domain 28; ADAM23; | | |
| | | | EMDCII; MDC-LM; MDC-LS; MDC-Lm; | | |
| | | | MDC-Ls; MDCL; eMDCII) | | |
| 1 | 0 | 0 | ANGPT2 | 0.003 | 2.1 |
| 1 | 0 | 0 | LOC84664 (melanoma-associated chondroitin | 0.008 | 2.3 |
| | | | sulfate proteoglycan-like) | | |
| 1 | 0 | 0 | RNASE1 (ribonuclease, RNase A family, 1 | 0.001 | 2.9 |
| | | | (pancreatic); RIB1; RNS1) | | |
| 0 | 1 | 1 | HAS1 | 1 | 0.3 |
| 0 | 1 | 0 | SLC16A8 | 1 | 0.4 |
| 0 | 1 | 0 | CD164 | 1 | 1.0 |
| 0 | 1 | 0 | FSTL1 | 1 | 1.0 |
| 0 | 1 | 0 | IL8 | 1 | 1.0 |
| 0 | 1 | 0 | KTN1 | 1 | 1.0 |
| 0 | 1 | 0 | RBP4 | 1 | 1.1 |
| 0 | 1 | 0 | COL5A2 | 1 | 1.1 |
| 0 | 1 | 0 | TSPAN-3 | 1 | 1.1 |
| 0 | 1 | 0 | CD63 | 1 | 1.1 |
| 0 | 1 | 0 | IGFBP3 | 1 | 1.1 |
| 0 | 1 | 0 | PLEC1 | 1 | 1.1 |
| 0 | 1 | 0 | CXCL2 | 1 | 1.2 |
| 0 | 1 | 0 | GPR48 | 1 | 1.2 |
| 0 | 1 | 0 | FLJ20559 | 1 | 1.2 |
| 0 | 1 | 0 | LAMB1 | 1 | 1.3 |
| 0 | 1 | 0 | COL4A1 | 0.994 | 1.3 |
| 0 | 1 | 0 | TFP12 | 1 | 1.4 |
| 0 | 1 | 0 | ESR1 | 0.996 | 1.5 |
| 0 | 1 | 0 | SLC11A3 | 0.999 | 1.6 |
| 0 | 1 | 0 | EFNA1 | 1 | 1.6 |
| 0 | 1 | 0 | KLK13 | 1 | 2.5 |
| 0 | 1 | 0 | AB065858 | 1 | 3.1 |
| 0 | 1 | 0 | MMP7 | 0.987 | 3.4 |
| 0 | 1 | 0 | INHBB | 1 | 3.5 |
| 0 | 0 | 1 | PI3 (protease inhibitor 3, skin-derived | 1 | 0.4 |
| | | | (SKALP); WAP four-disulfide core domain 14; | | |
| | | | elafin precursor; elastase-specific inhibitor; | | |
| | | | skin-derived antileukoproteinase; ELAFIN; | | |
| | | | ESI; MGC13613; SKALP; WAP3; WFDC14) | | |

1. A method for identifying nucleic acid molecules, comprising
a) preparing at least one xenograft tumor from a cancer cell line;
b) obtaining nucleic acids expressed in the xenograft tumor;
b) obtaining the expression profile of said tumor by contacting nucleic acids expressed in the xenograft tumor with a microarray comprising nucleic acid probes for genes suspected of being expressed in the xenograft tumor; and
c) identifying nucleic acid molecules that are expressed in the xenograft tumor.

2. The method of claim 1, wherein the xenograft tumor is a human xenograft tumor.

3. The method of claim 2, wherein the human xenograft tumor is derived from adenocarcinoma cell lines selected from the group consisting of breast, colon, lung, ovarian and prostate.

4. The method of claim 1, further comprising identifying nucleic acids differentially expressed in the xenograft tumor relative to the parental cell lines from which the tumor was derived.

5. The method of claim 4, wherein the identifying comprises statistical analysis.

6. The method of claim 1, further comprising comparing the expression profiles of at least two xenograft tumors.

7. The method of claim 4, further comprising co-hybridizing all samples with a reference cDNA derived from at least one reference cell line.

8. The method of claim 4, wherein the nucleic acid molecules that are differentially expressed are selected from the group consisting of single-stranded DNA, double-stranded DNA, single-stranded RNA, and double-stranded RNA.

9. A microarray, comprising nucleic acid probes for known genes encoding secreted proteins, putative genes encoding secreted proteins; known genes encoding cell surface proteins, and putative genes encoding cell-surface proteins, wherein said genes are classified, and wherein the classification distribution is
behavior, about 1%;
adhesion, about 6%;
recognition, about 3%;
cell-cell signaling, about 8%;
response to external stimulus, about 10%;
signal transduction, about 30%;
cell growth and maintenance, about 22%;
cell death, about 2%;
development, about 9%; and
physiological processes, about 9%.

10. The microarry of claim 9, further comprising probes for positive and negative controls.

11. The microarray of claim 9, comprising 3531 nucleic acid probes.

12. The microarray of claim 11, comprising 1057 nucleic acid probes for genes encoding secreted proteins, and 1338 nucleic acid probes for genes encoding G-protein coupled receptors (GPCR).

13. The microarray of claim 9, wherein each probe is present in more than one copy.

14. The microarry of claim 9, wherein each probe is a 60-mer.

15. A microarray, comprising nucleic acid probe molecules specific for genes selected from the group consisting of the genes listed in Table 1, the genes listed in Table 2, and the genes listed in Table 1 and Table 2.


SEQUENCE LISTING (26 sequences)

SEQ ID NO: 1 - DNA, 36 bp, Artificial
  primer
cctggcacccagcacaatcctggcacccagcacaat

SEQ ID NO: 2 - DNA, 40 bp, Artificial
  primer
gccgatccacacggagtactgccgatccacacggagtact

SEQ ID NO: 3 - DNA, 52 bp, Artificial
  primer
agcaaaatgaaagagaacatgaaatgagcaaaatgaaagagaacatgaaatg

SEQ ID NO: 4 - DNA, 52 bp, Artificial
  primer
ttcaaccaataaactgagaaagaagcttcaaccaataaactgagaaagaagc

SEQ ID NO: 5 - DNA, 52 bp, Artificial
  primer
attttgggctcttagcttagtctgttattttgggctcttagcttagtctgtt

SEQ ID NO: 6 - DNA, 44 bp, Artificial
  primer
ggttacaacggtgtttgcatgaggttacaacggtgtttgcatga

SEQ ID NO: 7 - DNA, 34 bp, Artificial
  primer
atgtggccgttccctgcatgtggccgttccctgc

SEQ ID NO: 8 - DNA, 48 bp, Artificial
  primer
tcttctctgtccacaagtttccagtcttctctgtccacaagtttccag

SEQ ID NO: 9 - DNA, 44 bp, Artificial
  primer
attcctgagccagtgtcaggagattcctgagccagtgtcaggag

SEQ ID NO: 10 - DNA, 48 bp, Artificial
  primer
ctgtctttccactgtgggtgtaagctgtctttccactgtgggtgtaag

SEQ ID NO: 11 - DNA, 40 bp, Artificial
  primer
ggcaaagccaaaatagctccggcaaagccaaaatagctcc

SEQ ID NO: 12 - DNA, 42 bp, Artificial
  primer
ctgccacatgacgagggatatctgccacatgacgagggatat

SEQ ID NO: 13 - DNA, 46 bp, Artificial
  primer
ctagtctttgggaaacgggttgtctagtctttgggaaacgggttgt

SEQ ID NO: 14 - DNA, 40 bp, Artificial
  primer
aaatccaacacggcaaaggaaaatccaacacggcaaagga

SEQ ID NO: 15 - DNA, 44 bp, Artificial
  primer
gctttccactccaattcacacagctttccactccaattcacaca

SEQ ID NO: 16 - DNA, 48 bp, Artificial
  primer
cctggccttgattctgttaatacccctggccttgattctgttaatacc

SEQ ID NO: 17 - DNA, 40 bp, Artificial
  primer
tccccagctgtcttatggcttccccagctgtcttatggct

SEQ ID NO: 18 - DNA, 36 bp, Artificial
  primer
cagcacggaaattcctcccagcacggaaattcctcc

SEQ ID NO: 19 - DNA, 40 bp, Artificial
  primer
catgctcgagtcatcagccacatgctcgagtcatcagcca

SEQ ID NO: 20 - DNA, 40 bp, Artificial
  primer
tttcactgccccctttgtgttttcactgccccctttgtgt

SEQ ID NO: 21 - DNA, 38 bp, Artificial
  primer
aagctctggaggccgactgaagctctggaggccgactg

SEQ ID NO: 22 - DNA, 38 bp, Artificial
  primer
ggccttgtcatcccgactcggccttgtcatcccgactc

SEQ ID NO: 23 - DNA, 42 bp, Artificial
  primer
tacagcatctctcggacggagtacagcatctctcggacggag

SEQ ID NO: 24 - DNA, 42 bp, Artificial
  primer
ggtgctattgaaagccttgcaggtgctattgaaagccttgca

SEQ ID NO: 25 - DNA, 42 bp, Artificial
  primer
ccctaagtgacccggacacttccctaagtgacccggacactt

SEQ ID NO: 26 - DNA, 46 bp, Artificial
  primer
cgttatatacacaggcggtgatgcgttatatacacaggcggtgatg